(19) United States
(12) Patent Application Publication
(10) Pub. No.: US 2012/0164132 A1
(43) Pub. Date: Jun. 28, 2012

(54) Anticancer and immunomodulating molecules and fractions containing said molecules, and process for preparing said fractions and said molecules from fermented vegetal material, and their uses

(76) Inventors: Mate Hidvegi, Budapest (HU); Gyula Bencze, Budapest (HU); Gyorgy Keri, Budapest (HU); Laszlo Orfi, Budapest (HU); Darryl Pappin, Boxborough, MA (US); Nicholas K. Tonks, Huntington, NY (US); James Watson, Cold Spring Harbor, NY (US)

(21) Appl. No.: 13/135,616

(22) Filed: Jul. 11, 2011

Related U.S. Application Data
(60) Provisional application No. 61/400,787, filed on Aug. 2, 2010.

(51) Int. Cl. A61K 36/899; A61P 37/00; A61P 3/00; A61P 35/00

(52) U.S. Cl. 424/123; 424/115

(57) ABSTRACT

Biologically active substances and fractions of wheat germ ferment/fermented wheat germ extract (FWGE), including formulation A250, the processes for their production, the pharmaceutical preparations containing them, and their uses.

### PRIORITY

This application claims priority to a provisional United States patent application filed Aug. 2, 2010 under application Ser. No. 61/400,787, the entire disclosure of which is hereby incorporated herein by reference.

### BACKGROUND OF THE INVENTION

1. Field of the Invention

The present disclosure relates to a series of natural and synthetic compounds and molecular mixtures useful as medicaments having anti-cancer, anti-metastatic, cancer-preventing, anti-inflammatory, immune-modulatory, metabolic-regulatory, cardiovascular-protective and anti-aging properties, and may be utilized as dietary supplements, and medical or dietary foods, and drugs or pharmaceuticals, and a process for producing the same, in particular, physiologically active compounds and molecular mixtures derived from wheat germ ferment, or fermented wheat germ extract.

2. Background Information

In The group of pharmaceutical and food-supplements extracted from fermented wheat germ has long been associated with stimulating the mammalian immune system. Said group has also been associated with anti-cancer and metabolic regulatory-related dietary supplements. The current disclosure describes a process for producing the active ingredient in said group and to pharmacological compositions comprising the same.

In Hungary, during the early 1990's, a proprietary fermented wheat germ extract (FWGE) with potent anti-cancer, anti-metastatic, cancer-preventing, anti-inflammatory, immune-modulatory, metabolic-regulatory, cardiovascular-protective and anti-aging properties was discovered by one of us (M. H.) (See e.g. Boros L G, et al. (2005): Fermented wheat germ extract (Avemar) in the treatment of cancer and autoimmune diseases. Ann N Y Acad Sci. 1051:529-42; Telekes A, et al. (2005): Fermented wheat germ extract (Avemar) inhibits adjuvant arthritis. Ann N Y Acad Sci. 1110:348-61; Pelletier M (2008): Unplugging cancer's power supply. Anti-aging Med News (Summer issue) 188-90; Iyer A, Brown L (2009): Fermented wheat germ extract (Avemar) in the treatment of cardiac remodeling and metabolic symptoms in rats. Evid Based Complement Alternat Med. doi:10.1093/ecam/nep090; Cassileth B R, et al. (2010): Wheat germ extract. In: Herb-drug interactions in oncology. Memorial Sloan-Kettering Cancer Center. Peoples' Medical Publishing House-USA, Shelton, Conn. p. 694-6).

Since its discovery, there have been several patents submitted on FWGE, such as: Hidvegi M, et al: Immunostimulatory and metastasis inhibiting fermented vegetal material. (PCT/HU98/00077; WO 99/08694. Priority date: Aug. 13, 1997). Hidvegi M: Pharmaceutical composition containing a fermented, dehydrated material with amorphous crystalline structure. (PCT/HU2010/000025; WO 2010/100514. Priority date: Mar. 3, 2009). Hidvegi M, et al: Fractions of wheat germ ferment. (PCT/HU2010/000026; WO 2010/100515 A2. Priority date: Mar. 6, 2009). The Regents of the University of California, et al: Fermented wheat germ proteins (FWGP) for the treatment of cancer. (PCT/US2010/035656; WO 2010/135580 A2. Priority date: May 20, 2009).

Currently, FWGE is manufactured in the United States and in the EU (in Hungary and Germany) by fermenting wheat germ, a by-product of wheat milling, in aqueous medium in the presence of Saccharomyces cerevisiae, and by drying the fermentation liquid. In the world, FWGE-containing different products for human use have widely been distributed under various trade names, such as products containing freeze-dried FWGE (99.6%) with added inert flavoring materials (Oncomar, Oncomar-Avemar Lyophilisate, Avé, ULTRA, Oncomar Capsules); and products containing spray-dried FWGE with significant amounts of additives, such as maltodextrin, silicon dioxide and fructose, with added flavoring materials (Avemar, Avé, WGE Avemar MSC), etc.

In countries of the European Community, in accordance with EU directive 1999/21/EC, FWGE-containing products have been notified as dietary foods for special medical purposes for cancer patients while, in the USA, due to the different regulatory environment, the same preparations have been marketed as dietary supplements with claims of supporting normal immune function and normal cell metabolism.

Large amount of basic and applied (clinical) research work has been dedicated to the deeper understanding of the mechanisms by which FWGE exerted its many-folded biological actions. According to PubMed.gov, there have been over thirty peer-reviewed papers published on FWGE, as assessed on Jun. 27, 2011 (http://www.ncbi.nlm.nih.gov/pubmed?term=avemar).

FWGE inhibits the activities of several enzymes involved in de novo nucleic acid synthesis and in supplying the deoxyribonucleoside triphosphates (dNTPs) pool required for DNA replication (http://www.cancer.gov/drugdictionary?CdrID=529839). The extract inhibited ribonucleotide reductase (RR) (Illmer C, et al. (2005): Immunologic and biochemical effects of the fermented wheat germ extract Avemar. Exp Biol Med. 230:144-9), the key enzyme of de novo DNA synthesis. Among several other mechanisms responsible for the anticancer effect of FWGE (Telekes A, Raso E (2007): Changes in the kinase expression panel of K562 human leukemia after Avemar treatment. J Clin Oncol. 25(18S):14143), it has been stated that the decreased oxidative ribose synthesis of cancer cells caused by this extract might also limit the metabolic needs of tumor cells for the conversion of ribonucleotides to dNTPs, which are the precursors of DNA synthesis. The responsible enzyme, RR was demonstrated to be significantly up-regulated in tumor cells to meet the increased need for dNTPs of these rapidly proliferating cells. It was able to demonstrate that the RR in situ activity of tumor cells could be inhibited by FWGE in a concentration-dependent manner and, the decrease of RR in situ activity reached a maximum, and higher dosage of the extract was not able to intensify the observed effect (Saiko P, et al. (2007): Avemar, a nontoxic fermented wheat germ extract, induces apoptosis and inhibits ribonucleotide reductase in human HL-60 promyelocytic leukemia cells. Cancer Lett. 250:323-8). This might be the reason for the fact that FWGE demonstrated no signs of additional toxicity in various studies, whereas other inhibitors of RR, such as some chemotherapeutic drugs, exerted dose-limiting toxic side effects when applied to humans. FWGE also induces caspase-3-mediated inactivation of poly(ADP)ribose polymerase (PARP) (Comin-Anduix B, et al. (2002): Fermented wheat germ extract inhibits glycolysis/pentose cycle enzymes and induces apoptosis through poly(ADP-ribose) polymerase activation in Jurkat T-cell leukemia tumor cells. J Biol Chem. 277:46408-14), a key enzyme in DNA repair that is over-expressed in many cancers; cleavage of PARP prevents DNA repair and induces apoptosis. In tumor cells, not in healthy cells, FWGE induced a dose-dependent cytotoxic effect and induced typical pattern of apoptotic cell death. Clinically relevant doses of FWGE induced a significant decrease of S-phase fraction over time and an increase of sub-G1-peak as further indication of an apoptotic cell population (Lee S N, et al. (2005): Cytotoxic activities of fermented wheat germ extract (FWGE) on human gastric carcinoma cells by induction of apoptosis. J Clin Oncol. 23(165):4254). To exert its cytotoxic effects, FWGE inhibited the activity of two key enzymes of the pentose cycle, glucose-6-phosphate dehydrogenase and transketolase, and thereby regulated the carbon flow in the pentose cycle in cancers. Concomitantly, the two key enzymes in the regulation of the glycolytic flux, lactate dehydrogenase and hexokinase, were selectively inhibited in cancer cells, as well (Comin-Anduix B, et al. (2002): Fermented wheat germ extract inhibits glycolysis/pentose cycle enzymes and induces apoptosis through poly(ADP-ribose) polymerase activation in Jurkat T-cell leukemia tumor cells. J Biol Chem. 277:46408-14). Concomitantly, dose-dependent decrease of glucose consumption and of ribosomal RNA-synthesis through non-oxidative steps of the pentose cycle were observed in FWGE treated tumors (Boros L G, et al. (2001): Wheat germ extract decreases glucose uptake and RNA ribose formation but increases fatty acid synthesis in MIA pancreatic adenocarcinoma cells. Pancreas 23:141-7).

Besides its direct tumor growth inhibitory effect, FWGE inhibited the development and growth of tumor metastasis on its own or synergistically in the combination with anticancer drugs (Hidvegi M, et al. (1998): Effect of Avemar and Avemar+vitamin C on tumor growth and metastasis in experimental animals. Anticancer Res. 18:2353-8; Hidvegi M et al. (1999): MSC, a new benzoquinone-containing natural product with anti-metastatic effect. Cancer Biother Radiopharm. 14:277-89), and was able to overcome the resistance of tumor cells against 5-FU, a conventional chemotherapeutic drug (Saiko P, et al. (2009): Avemar, a nontoxic fermented wheat germ extract, attenuates the growth of sensitive and 5-FdUrd/Ara-C cross-resistant H9 human lymphoma cells through induction of apoptosis. Oncol Rep. 21:787-791). Interestingly, the immune-modulatory effects of FWGE could not be ascribed to 2,6-dimethoxy-p-benzoquinone alone, since it didn't restore healthy immune responses as compared to the complete formulation (Hidvegi, et al. (1999): Effect of MSC on the immune response of mice. Immunopharmacology 41:183-6). A potential component of Avemar's immune-modulatory properties is its ability to down-regulate the MHC class I proteins on tumor cells. This hinders the tumor cells' strategy to mimic themselves as normal cells in order to escape the immune defense and sensitizes them against natural killer (NK) cell surveillance (Fajka-Boja R, et al. (2002): Fermented wheat germ extract induces apoptosis and down-regulation of major histocompatibility complex class I proteins in tumor T and B cell lines. Int J. Oncol. 20:563-70).

Endothelial cells of the vasculature of human solid tumors are known to have decreased expression of intercellular adhesion molecule-1 (ICAM-1) compared to normal endothelial cell tissue, and this phenomenon can be considered a tumor-derived escape mechanism because the development of an efficient leukocyte infiltrate of the tumor is impaired. It has been shown that FWGE up-regulated the expression of ICAM-1 on tumor-derived endothelial cells and also potentiated the similar effect of the primary anticancer cytokine, tumor necrosis factor-alpha (Telekes A, et al. (2005): Fermented wheat germ extract (Avemar) inhibits adjuvant arthritis. Ann N Y Acad Sci. 1110:348-61).

A clear inhibition of experimental colon carcinogensis by FWGE in F-344 rats has also been reported. Using azoxymethane injections, FWGE treatment could significantly reduce the emergence of colon tumors, thus indicating a role of FWGE in the prevention of cancer (Zalatnai A, et al. (2001): Wheat germ extract inhibits experimental colon carcinogenesis in F-344 rats. Carcinogenesis 22:1649-52).

FWGE on its own significantly inhibited the growth of both estrogenic receptor positive (ER+) and estrogenic receptor negative (ER−) breast tumors. When applied in combination with endocrine drugs (tamoxifen, exemestane and anastrozol) for the treatment of ER+ breast cancers, FWGE systematically increased the efficacy of the drugs (Marcsek Z, et al. (2004): The efficacy of tamoxifen in estrogen receptor-positive breast cancer cells is enhanced by a medical nutriment. Cancer Biother Radiopharm. 19:746-53; Tejeda M, et al. (2007): Avemar inhibits the growth of mouse and human xenograft mammary carcinomas comparable to endocrine treatments. J Clin Oncol. 25(18S):21132) and, therefore the inclusion of FWGE into the treatment protocols of both ER+ and ER− breast cancers can be recommended.

In in vivo cancer studies, the dose-response curves of FWGE, in terms of progression-free and overall survivals, are bell-shaped. This peculiar phenomenon is considered a “biological fingerprint” of FWGE (see: Farkas E (2006). Use of the fermented wheat germ extract (Avemar) in family medicine practice. Medicus Universalis 39(1):19-31 (In Hungarian)).

FWGE improved survival, and reduced new recurrences and metastases in colorectal cancer patients (Jakab F, et al. (2000): First clinical data of a natural immunomodulator in colorectal cancer. Hepatogastroenterology 47:393-5). When used in a study of 170 post-surgical colorectal cancer patients, also receiving standard of care therapy such as chemotherapy, and/or radiation, addition of FWGE reduced new recurrences by 82%, metastases by 67%, and deaths by 62%, compared to use of radiation and chemotherapy alone (Jakab F, et al. (2003): A medical nutriment has supportive value in the treatment of colorectal cancer. Br J. Cancer. 89:465-9). It also prolonged time to progression, i.e. the time it took for cancer to become measurably active again after primary therapy (surgery) and adjuvant therapy (chemotherapy and/or radiation treatment). In pediatric cancer patients with various cancer types, treated by high-dose chemotherapies, FWGE substantially reduced the risk of febrile neutropenia, primarily by boosting immune system cell populations and activity (Garami M, et al. (2004): Fermented wheat germ extract reduces chemotherapy induced febrile neutropenia in pediatric cancer patients. J Pediatr Hematol Oncol. 26:631-5). In pre-clinical tests specifically looking at immune effects, FWGE accelerated recovery of immune function following radiation and chemotherapy, inhibited immune suppression, improved NK cell recognition of target cells, and supported normal immune system function that helps white blood cells to cross through blood vessel walls and into tumors (Johanning G L, Wang-Johanning F (2007): Efficacy of a medical nutriment in the treatment of cancer. Altern Ther Health Med. 13:56-63). In a randomized, pilot, phase II clinical trial, the efficacy of dacarbazine-based adjuvant chemotherapy on survival parameters of melanoma patients was compared to that of the same treatment supplemented with a 1-year long administration of FWGE. At the end of an additional 7-year-long follow-up period, highly significant differences in both progression-free and overall survival in favor of the FWGE patients were found. It was concluded that the inclusion of FWGE into the adjuvant protocols of high-risk skin melanoma patients is highly recommended (Demidov et al. (2008): Adjuvant fermented wheat germ extract (Avemar) nutraceutical improves survival of high-risk skin melanoma patients: A randomized, pilot, phase II clinical study with a 7-year follow-up. Cancer Biother Radiopharm. 23:477-82.). In an oral cancer study, FWGE used as supportive therapy for patients undergoing standard anticancer therapies for locally advanced squamous cell carcinoma of the mouth, FWGE reduced the risk of cancer progression by 85%. In 2008, the National Committee of Oral Diseases of Health and Welfare Hungary, issued the statement that FWGE is an integral part of the treatment protocols of oral cancer patients (Barabas J, Nemeth Z (2006): Recommendation of the Hungarian Society for Face, Mandible and Oral Surgery in the indication of supportive therapy with Avemar. Orv Hetil. 147:1709-11 (in Hungarian)). FWGE has also been shown to possess supportive value in the treatment of ovarian cancer, gastric cancer, thyroid cancer, non-Hodgkin's lymphoma, chronic myelogenous leukemia, and multiple myeloma. Regression in patients with advanced hepatocellular carcinoma who have been taking FWGE on a continuous basis has been observed. Interestingly, regression in skeletal metastatic lesions has also been reported in last-stage breast, prostatic, and non-small-cell lung cancer patients (Boros L G, et al. (2005): Fermented wheat germ extract (Avemar) in the treatment of cancer and autoimmune diseases. Ann N Y Acad Sci. 1051:529-42).

In pre-clinical tests to determine whether FWGE might interfere with conventional anti-cancer therapies, it was concluded that the compound could be included into the chemotherapeutic protocols for the treatment of cancer patients (Szende B, et al. (2004): Effect of simultaneous administration of Avemar and cytostatic drugs on viability of cell cultures, growth of experimental tumors, and survival tumor-bearing mice. Cancer Biother Radiopharm. 19:343-9; Voigt W, et al. (2009): Promising cytotoxic activity profile of fermented wheat germ extract (Avemar) in human cancer cell lines. Eur J Cancer S7:110; Mueller T, et al. (2011): Promising cytotoxic activity profile of fermented wheat germ extract (Avemar) in human cancer cell lines. J Exp Clin Cancer Res. 30:42. doi:10.1186/1756-9966-30-42). In another study, therapeutic effects of some conventional treatments used in combination with FWGE were increased, e.g., reduced metastasis (Jakab F, et al. (2003): A medical nutriment has supportive value in the treatment of colorectal cancer. Br J Cancer 89:465-9), and in some cases those effects were accompanied by lessened frequency and severity of common side effects of conventional treatments, such as nausea, fatigue, weight loss and immune suppression (Demidov L V, et al. (2008): Adjuvant fermented wheat germ extract (Avemar) nutraceutical improves survival of high-risk skin melanoma patients: A randomized, pilot, phase II clinical study with a 7-year follow-up. Cancer Biother Radiopharm. 23:477-82). In quality of life clinical studies, FWGE improved the quality of life of cancer patients (Sukkar G S, et al. (2008): A multicentric prospective open trial on the quality of life and oxidative stress in patients affected by advanced head and neck cancer treated with a new benzoquinone-rich product derived from fermented wheat germ (Avemar). Mediterr J Nutr Metab. 1:37-42).

The relatively large recommended single daily dose of FWGE (approximately 5.5 g pure extract for an average adult person) makes the suggested quantity difficult to consume particularly for head-and-neck cancer patients with dysphagia. Over this problem, the product is hygroscopic and, the spray-dried FWGE-containing formulations have unpleasant taste and smell, which also limits their widespread use among cancer patients undergoing chemotherapy, which can frequently cause nausea and, also due to their often unpalatable organoleptic characteristics, for pediatric cancer patients, the spray-dried FWGE-containing products are generally burdensome when consumed.

According to a recent invention (PCT/HU2010/000026) by some of us (M. H., G. B., G. K., L. O., as the Hungarian Research Group, HRG), FWGE had successfully been taken to three principal fractions A2, F1 and E, from which F1 turned out to be as inactive waste, while principal fractions A2 and E comprised all of the biological activity of the original FWGE. Thus, the required daily dose of FWGE could be reduced by a significant extent and, the previous problems associated with the hygroscopic nature and the unfavorable organoleptic characteristics of the whole extract could be entirely eliminated, as well. Though, the single daily dose, necessary to take, still remained relatively high (>3 g), and therefore burdensome to consume, particularly for some specialty consumer groups. This latter mentioned weakness of the said disclosure (PCT/HU2010/000026) represented a challenge for the scientific community.

The patent application submitted by researchers of The University of California (PCT/US2010/035656), which provided compositions containing anti-cancer polypeptides isolated from the whole FWGE, was also an interesting development of the art, however this application suffered from a serious shortcoming, notably, a great variety of the valuable, physiologically active molecules, the non-proteinaceous (i.e. not polypeptides or peptides) compounds of FWGE, had been lost by that invention.

It is common knowledge that FWGE contains two, wheat germ-specific, well-known sorts of biologically active compounds: lectins (WGA, wheat germ agglutinin) and methoxy-substituted benzoquinones (DMBQ, 2,6-dimethoxy-p-benzoquinone; MBQ, 2-methoxybenzoquinone). Though, industrially manufactured FWGE has been standardized to the DMBQ content and to the high-performance liquid chromatography (HPLC) fingerprint chromatogram of the benzoquinones (see e.g. Heimbach J T, et al. (2007): Safety studies regarding a standardized extract of fermented wheat germ. Int J. Toxicol. 26:253-9), it had early become evident that the benzoquinones on their own were not the real, or at least not the sole, active ingredients of the preparation (see e.g. Hidvegi M, et al. (1999): Effect of MSC on the immune response of mice. Immunopharmacology 41:183-6). Although, WGA had soon been proposed the bioactive component of FWGE (Baintner K (1999): A wheat germ preparation and its possible action. Orv Hetil. 140:1141-3 (in Hungarian)), later on, it was shown that the lectin components, similarly to the benzoquinones, were not the real and physiologically significant active molecules of the extract (see e.g. Fajka-Boja R, et al. (2002): Fermented wheat germ extract induces apoptosis and down-regulation of major histocompatibility complex class I proteins in tumor T and B cell lines. Int J. Oncol. 20:563-70). Here, it also has to be noted that WGA has an unfavorable toxicity profile and, if administered intravenously, can even be lethal to mammals. We can also say that WGA could at least partly be responsible for the significant in vitro anti-proliferative efficacy of FWGE on a wide spectrum of cancer cell lines, found by several research groups (see e.g. Mueller T, et al. (2011): Promising cytotoxic activity profile of fermented wheat germ extract (Avemar) in human cancer cell lines. J Exp Clin Cancer Res. 30:42. doi:10.1186/1756-9966-30-42).

There are several problems, which can be associated with the lack of sufficient knowledge on the truly active components of FWGE. One of such problems is the difficulty of meeting the good manufacturing practice (GMP) requirements at the industrial production of the extract. Among others, GMP requires the implementation of a particular quality control system ensuring that the product complies with quality standards including those for potency, efficacy, homogeneity, etc. It is difficult and costly to meet these requirements with a product which is intended to be used by cancer patients but, has yet unidentified active components. For example, instead of quick and relatively cheap chemical tests to monitor the concentration of the active ingredients, one has to apply animal cancer experiments, which are cost- and time-consuming, to test the efficacy-adequacy of the product. With FWGE, due to the presence of WGA, it is also not enough to carry out in vitro tests of efficacy, because the in vitro anti-cancer effects of the lectins may stretch the outcomes of the quality control process. These types of uncertainties my also undermine any future drug approval applications of FWGE. We can thus say, that finding the active components or the physiologically relevant minimal mixture of the molecules (the “active complex” or the “active ingredient core”) responsible for the significant biological effects of FWGE is of greater importance than one could think.

In 2009, a research collaboration between the Cold Spring Harbor Laboratory and the HRG was initiated to identify and characterize the active constituents of FWGE (see Cold Spring Harbor Laboratory 2009 Annual Report. Cold Spring Harbor, N.Y., 2010. p. 102-103). The primary objective of the collaboration was to identify and characterize the above mentioned “active ingredient core” of FWGE thus, rendering the development of an easily applicable pharmaceutical formulation (e.g. a single daily pill or capsule, or shot, or intravenous drip or injection), which can exert equivalent or preferably higher physiological efficacy than the recommended single daily oral dose (5.5 g) of the whole extract, while representing no considerable burden to customers for whom the administration of the usual dose of FWGE or that of the principal fractions had been difficult or even impossible.

### Preliminary Discovery

First, the anti-cancer efficacies of the principal fractions A2 and E of FWGE, manufactured by the HRG according to the invention: PCT/HU2010/000026, have first been compared by us. The principal fraction A2 also contains the benzoquinones, and the principal fraction E also contains the WGA from the original FWGE. It was found that both principal fractions showed comparable and significant anti-proliferative activity against different human cancer cell lines. When however, the principal fractions were tested in human ovarian cancer cell lines and their non-tumorigenic but immortalized counterparts, A2 surprisingly showed great cell-killing selectivity, which is one of the most important characteristics of the sought-after anti-cancer preparations, while, E did not (see TABLE 1). Therefore A2 was chosen as the starting material for the biological assay-guided fractionation work treated in the present disclosure.

TABLE 1
The half maximal inhibitory concentration (IC₅₀) values, expressed as
mcg/ml, of FWGE and its principal fractions against human ovarian
cell lines.

| Cell lines | FWGE | A2 | E |
|---|---|---|---|
| OVCAR-5¹ | 258 | 168 | 256 |
| PA-1² | 281 | 204 | 135 |
| HOSE11-12³ | 323 | >750⁴ | 172 |

^{1,2}Human ovarian cancer cells;
³Immortalized, non-tumorigenic, non-transfected human ovarian cells;
⁴Estimated value: 7321 mcg/ml

### BRIEF DESCRIPTION OF THE DRAWINGS

FIG. 1 is an isolation chart of the disclosed fractions.

FIG. 2 shows the characteristic HPLC Fingerprint UV chromatogram (HPLC) of A250 at 254 nm produced from an industrial batch (AP) of the FWGE, manufactured in different production plants, in a different country, at a different time.

FIG. 3 shows the characteristic HPLC Fingerprint UV chromatogram of A250 at 254 nm produced from an industrial batch (AVG-c7) of the FWGE, manufactured in a different production plant in a different country at a different time.

FIG. 4 shows the characteristic HPLC Fingerprint UV chromatogram of A250 at 254 nm produced from an industrial batch (AVU) of the FWGE, manufactured in a different production plant in a different country at a different time.

FIG. 5 shows the UV chromatogram of A250, produced from an industrial batch (AVU) of the FWGE, (denoted as A250-AVU) at 254 nm, with the most characteristic peaks indicated by numbers.

FIG. 6 shows the UV-spectrum (HPLC) of A250-AVU at 6.6 min.

FIG. 7 shows the UV-spectrum (HPLC) of A250-AVU at 7.6 min.

FIG. 8 shows the UV-spectrum (HPLC) of A250-AVU at 8.1 min.

FIG. 9 shows the UV-spectrum (HPLC) of A250-AVU at 8.5 min.

FIG. 10 shows the UV-spectrum (HPLC) of A250-AVU at 8.9 min.

FIG. 11 shows the UV-spectrum (HPLC) of A250-AVU at 9.3 min.

FIG. 12 shows the UV-spectrum (HPLC) of A250-AVU at 9.4 min.

FIG. 13 shows the UV-spectrum (HPLC) of A250-AVU at 9.7 min.

FIG. 14 shows the UV-spectrum (HPLC) of A250-AVU at 10.9 min.

FIG. 15 shows the UV-spectrum (HPLC) of A250-AVU at 11.2 min.

FIG. 16 shows the UV-spectrum (HPLC) of A250-AVU at 11.4 min.

FIG. 17 shows the UV-spectrum (HPLC) of A250-AVU at 12.4 min.

FIG. 18 shows the anti-tumor effects of FWGE and A250 in sarcoma mice; A250, produced from two, industrially manufactured FWGE batches of different origin, was administered in two different doses.

FIG. 19 shows the biological functions associated with proteins significantly upregulated or downregulated by >2 SD (standard deviation) following treatment of B16 melanoma cells with A250 at 4, 12 and 24 hours.

### BRIEF SUMMARY OF THE INVENTION

The current disclosure teaches methods of treatment and/or prevention of cancer and/or immunological diseases and/or metabolic imbalances, characterized by administering to a patient an effective amount of the pharmaceutical preparation or pharmaceutical preparations containing one or more of the isolated and/or synthesized compounds from fraction A2 of wheat germ ferment, described in PCT/HU2010/000026 (Hidvegi M, et al: Fractions of wheat germ ferment. International Patent Application).

Also disclosed are uses of compounds, isolated and/or synthesized from fraction A2 of wheat germ ferment, described in PCT/HU2010/000026, for the production of preparations, such as dietary supplement, medical food or dietary food for special medical purpose, herbal medicine, drug for mammals, respectively, formulated in delivery modalities of tablets, coated tablets, dragées, coated dragées, granules, sachets, capsules, solution, suspension, emulsion, spray, suppository, ointment, patch, liposome, having anti-cancer and/or immune-modulatory and/or metabolism-regulatory properties.

Also disclosed is a method of isolating and/or synthesizing a compound of formulation A250 having anti-cancer, immune-modulatory, metabolism-regulatory, dietary supplement, medical food, dietary food for special medical purpose, herbal medicine, drug for mammals properties, respectively. This non-toxic compound is a well-defined fraction of principal fraction A2 of FWGE, described in PCT/HU2010/000026, representing approximately 2.5-3% of the whole extract.

Also disclosed is a method of quality control for standardizing the production of fermented wheat germ extract, and standardizing the products containing the same extract, to the content of compound of formulation A250, which is a well-defined fraction of principal fraction A2 of fermented wheat germ extract, described in PCT/HU2010/000026.

Also disclosed is the description of the compound of formulation A250 itself.

Also disclosed is a method of synthesizing of compounds from fraction A2 of wheat germ ferment, described in PCT/HU2010/000026, identified as A2-NWS and A250-NSB.

Also disclosed is a method of synthesizing a set of compounds which are fractions of fermented wheat germ extract derivatives, identified as A2-80, A2KL, A2KLI−, A2KLI+, A2KLID, A2KLIDW, A2KLID50, A2KLIDM, A2KLIDZW, A2KLIDZKP, A2KLIDZF, A2KLIDZWS, obtained by fractionation of wheat germ ferment, which is obtained by fermenting wheat germ in aqueous medium in the presence of Saccharomyces cerevisiae, and by concentrating and/or dehydrating the fermentation liquid.

### DETAILED DESCRIPTION

In the following description, for purposes of explanation, specific numbers, materials and configurations are set forth in order to provide a thorough understanding of the present invention. However, it will be apparent to one skilled in the art that the present invention may be practiced without the specific details. In other instances, well-known features are omitted or simplified in order not to obscure the present invention. Furthermore, for ease of understanding, certain method steps are delineated as separate steps, however, these separately delineated steps should not be construed as necessarily order dependent in their performance.

A250 is a well-defined fraction of the fermented wheat germ extract. This fermented wheat germ extract has been the subject of previous patents such as Hidvegi (PCT/HU2010/000025). A250 represents approximately 2.5-3% of the whole extract, which has been proven to have anti-cancer, immune-modulatory and metabolic-regulatory properties. According to the present invention, the biologically active molecules of the whole extract are successfully concentrated into A250.

A250 can be manufactured both in the laboratory and on an industrial scale and has a unique characteristic HPLC fingerprint chromatogram. A surprising and important observation of A250 is that regardless of the physical differences among different batches of the FWGE manufactured in various manufacturing facilities, in various countries and different times, all of the batches contained very similar concentrations of A250 with very similar amounts of biological activity. It is also a surprising and important observation of A250 that in the animal cancer experiment carried out with this compound (see Example-7), it showed the same type of that peculiar bell-shaped dose-response attribute, in terms of disease progression (tumor growth) and overall survival, as has been previously shown by the intact FWGE only. In summary therefore, it can be said that A250 can be considered by nature an “active complex”, or with other words, an “active ingredient core”, a significant common trait of all fermented wheat germ extracts manufactured worldwide.

In the current disclosure, the patent entitled Fractions of wheat germ ferment and, submitted by Hidvegi et al. (PCT/HU2010/000026) describes fractions obtained from wheat germ ferment which preserve the anti-cancer and immune-modulating efficacy of the original extract, or are more efficient but, are not hygroscopic and have better organoleptic characteristics.

The present invention isolates the fraction, denoted as A2, according to Example 1 of the patent submission PCT/HU2010/000026, with a modification, namely the methanolic extract is deep-frozen (−20- to −80 degrees Celsius) overnight, and the precipitate cold-filtered (by use of a 10-20 micrometer filter) and separated. The precipitate, denoted as A2-80, has shown no significant activity against cancer cells. (See Example-1) The obtained methanolic solution is evaporated, preferably under vacuum and, a honey-like material (A2) with reddish brown color is the result.

A2 shows highly significant anti-cancer activity against human cancer cell lines but not against healthy (non-transfected) cells. (See Preliminary Discovery sub-chapter of Background of the Invention.) A2 is dissolved in 5-times quantity (w/v) of methanol, and proportionally, chloroform is added to this mixture in the following ratio: methanol solution: added chloroform-5:1, 5:2, 5:3, 5:4, 5:7, 5:10, 5:15, 5:25. (That is, to the methanolic A2 solution first 1 part of chloroform is added, after another 1 part of chloroform, after another 1 part of chloroform, after another 1 part of chloroform, after another 3 parts of chloroform, after another 3 parts of chloroform, after another 5 parts of chloroform, and finally another 10 parts of chloroform are added.)

After every step, the mixture is filtered and the undissolved precipitate is separated. The resulting methanol-chloroform solution is evaporated, and also a reddish, honey-like material (A2KL) is produced. A2KL is dissolved in 5-times quantity (w/v) of isopropanol. The undissolved material (A2KLI−) is filtered out. If A2KLI− is dried (preferably by diisopropyl ether), a light-brown powder is the result. The remaining material in the isopropanol solution is denoted as A2KLI+.

Diisopropyl ether is added to this solution in the following ratio: isopropanol solution:added diisopropyl ether-5:2, 5:4, 5:7, 5:10. After every step, the mixture is filtered and the insoluble precipitate is separated. The resulting isopropanol-diisopropyl ether solution is evaporated, and a reddish-brownish oily material (A2KLID) is produced. A2KLID is emulsified in 10-times quantity of water, and the emulsion is flushed through a solid-phase extraction (SPE) adsorbent (e.g., Waters Oasis HLB Cartridge) with the use of injection/suction. First, an oily fraction (A2KLIDW) is eluted. After this, the column is washed with water thus, completely removing all of A2KLIDW. Following this step, the column is washed with 50% methanol several times. The eluted fractions are united, and dried under vacuum and, dried with the use of diisopropyl ether. The resulting dark-red powder (A2KLID50) could be readily dissolved in water. The yield of A2KLID50 is 1.5-3% of the original fermented wheat germ extract (calculated on dry matter basis).

It is noted that the original fermented wheat germ extract, or the A2 fraction, or other fractions could be refined by the above mentioned SPE method, respectively. Finally, the column is washed with 100% methanol several times, the eluted fractions are united, and dried under vacuum and, dried with the use of diisopropyl ether. The resulting dark-red powder is denoted as A2KLIDM. For the sake of simplicity, A2—after dissolved in water—could also be directly flushed through the column.

In another separation sequence, first A2 is dissolved in water. The solution is filtered through a 0.2 micron sterile filter. The water-insoluble residual material, denoted as A2-NWS, is collected. The filtered solution is adsorbed on a solid phase, preferably flushed through an appropriate SPE adsorbent-containing equipment. More preferably, the said equipment is a column. The column is washed with water and, after drying the aqueous solution, a fraction, denoted as A250-NSB, is obtained. After this step, the column is washed with 100% methanol and, the methanolic solution is dried. A dark red, reddish brown powder, denoted as A250 is obtained. The yield of A250 is about 2.5-3% of the original FWGE.

Among other, A250 can be characterized by its characteristic HPLC fingerprint chromatogram. For taking the fingerprint, A250 is dissolved in DMSO in 25 mg/ml final concentration. The solvent is sonicated, mixed by vortex and centrifuged before injection.

Instrument: Waters Pump Control; Waters Fluidics Organizer; Waters 2998 PDA Detector; Waters 2767 Sample Manager; Parameters: Solvent A: Water+0.05% formic acid; Solvent B: Acetonitrile+0.05% formic acid. Column: Luna C18(2); 150×4.6 mm; 5 mcm, 100 A (Phenomenex); Injection volume (mcl)-20.00.

Gradient:

| Time (min) | | Flow Rate (ml/min) | % A | % B | Curve |
|---|---|---|---|---|---|
| 1. | NaN | 2.00 | 95.0 | 5.0 | NaN |
| 2. | 1.00 | 2.00 | 95.0 | 5.0 | 6 |
| 3. | 15.00 | 2.00 | 70.0 | 30.0 | 6 |
| 4. | 15.50 | 2.00 | 5.0 | 95.0 | 6 |
| 5. | 17.50 | 2.00 | 5.0 | 95.0 | 6 |
| 6. | 18.00 | 2.00 | 95.0 | 5.0 | 6 |
| 7. | 20.00 | 2.00 | 95.0 | 5.0 | 6 |

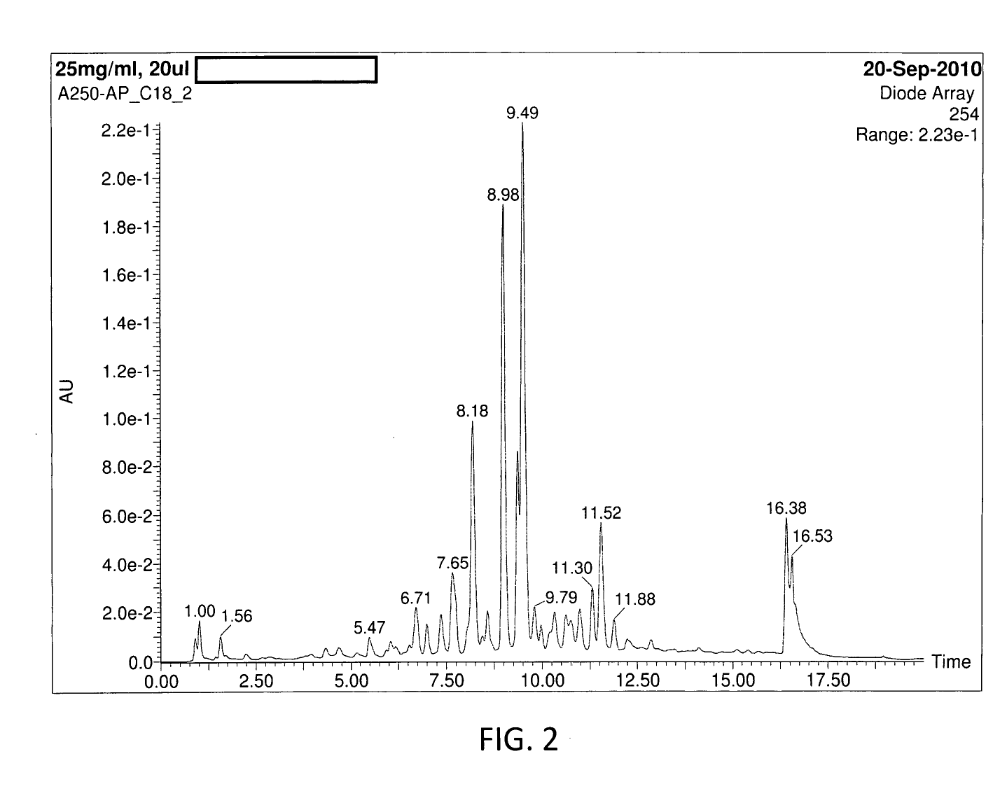

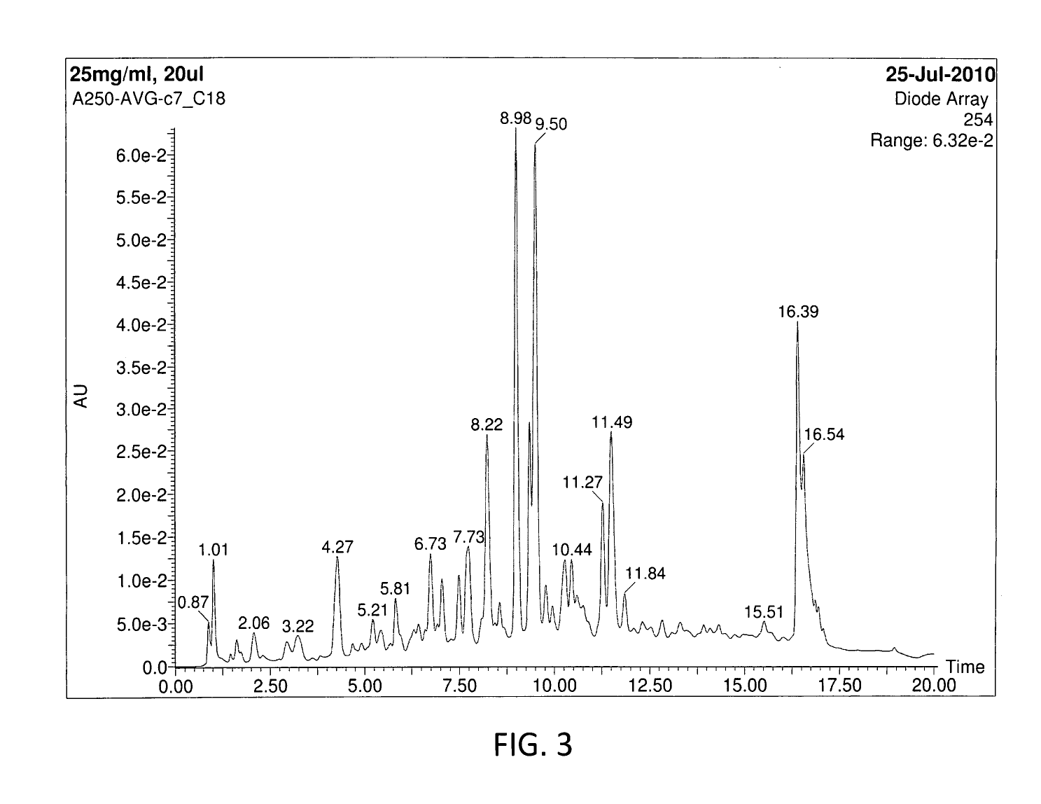

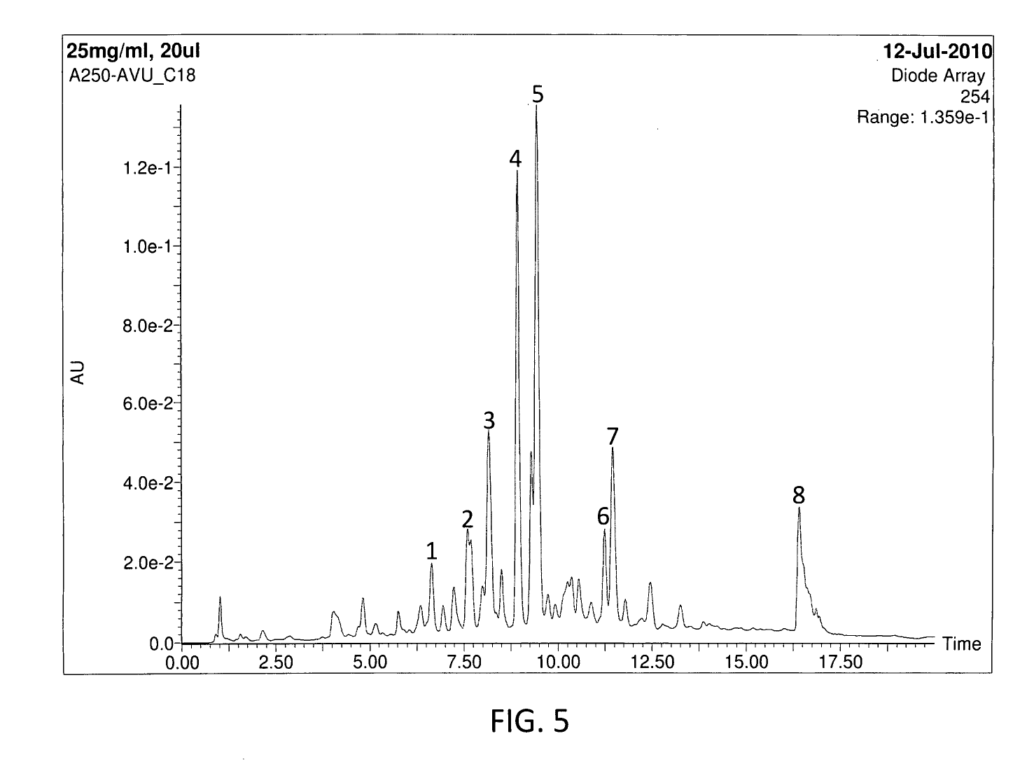

Characteristic HPLC fingerprint chromatograms of A250 are shown in FIGS. 2, 3 and 4. Retention times and relative retention quotients of the most characteristic peaks, indicated by numbers, as represented in FIG. 5, are shown in TABLE 2.

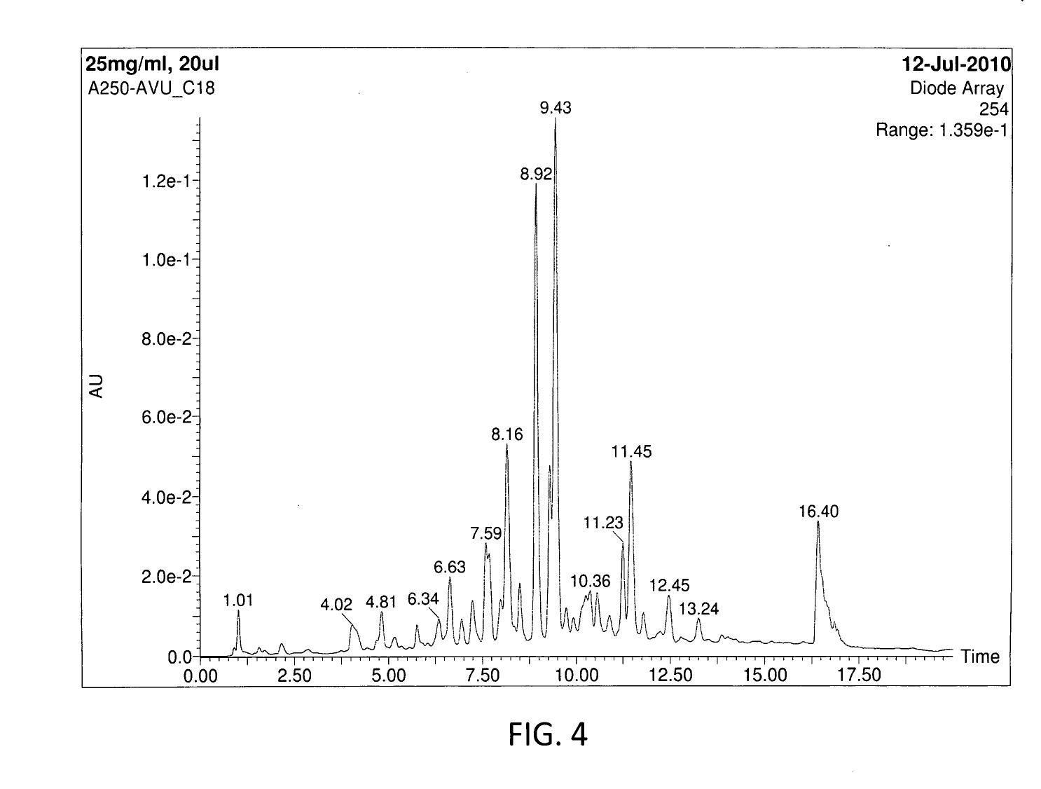

TABLE 2
Retention times (RT) and relative retention quotients of
the most characteristic peaks, indicated by numbers, on a
characteristic HPLC fingerprint chromatogram of A250, as
represented in FIG. 4. (AVU, c7 and AP are A250 samples manufactured
from different industrial batches of FWGE.)

| RT | AVU | c7 | AP | Mean | SD |
|---|---|---|---|---|---|
| Peak 1 | 6.63 | 6.73 | 6.71 | 6.69 | 0.05 |
| Peak 2 | 7.59 | 7.73 | 7.65 | 7.66 | 0.07 |
| Peak 3 | 8.16 | 8.22 | 8.18 | 8.19 | 0.03 |
| Peak 4 | 8.92 | 8.98 | 8.98 | 8.96 | 0.03 |
| Peak 5 | 9.43 | 9.5 | 9.49 | 9.47 | 0.04 |
| Peak 6 | 11.23 | 11.27 | 11.3 | 11.27 | 0.04 |
| Peak 7 | 11.45 | 11.49 | 11.52 | 11.49 | 0.04 |
| Peak 8 | 16.4 | 16.39 | 16.38 | 16.39 | 0.01 |

| RRT/AVU | 6.63 | 7.59 | 8.16 | 8.92 | 9.43 | 11.23 | 11.45 | 16.4 |
|---|---|---|---|---|---|---|---|---|
| 6.63 | 1.00 | 1.14 | 1.23 | 1.35 | 1.42 | 1.69 | 1.73 | 2.47 |
| 7.59 | | 1.00 | 1.08 | 1.18 | 1.24 | 1.48 | 1.51 | 2.16 |
| 8.16 | | | 1.00 | 1.09 | 1.16 | 1.38 | 1.40 | 2.01 |
| 8.92 | | | | 1.00 | 1.06 | 1.26 | 1.28 | 1.84 |
| 9.43 | | | | | 1.00 | 1.19 | 1.21 | 1.74 |
| 11.23 | | | | | | 1.00 | 1.02 | 1.46 |
| 11.45 | | | | | | | 1.00 | 1.43 |
| 16.4 | | | | | | | | 1.00 |

| RRT/c7 | 6.73 | 7.73 | 8.22 | 8.98 | 9.5 | 11.27 | 11.49 | 16.39 |
|---|---|---|---|---|---|---|---|---|
| 6.73 | 1.00 | 1.15 | 1.22 | 1.33 | 1.41 | 1.67 | 1.71 | 2.44 |
| 7.73 | | 1.00 | 1.06 | 1.16 | 1.23 | 1.46 | 1.49 | 2.12 |
| 8.22 | | | 1.00 | 1.09 | 1.16 | 1.37 | 1.40 | 1.99 |
| 8.98 | | | | 1.00 | 1.06 | 1.26 | 1.28 | 1.83 |
| 9.5 | | | | | 1.00 | 1.19 | 1.21 | 1.73 |
| 11.27 | | | | | | 1.00 | 1.02 | 1.45 |
| 11.49 | | | | | | | 1.00 | 1.43 |
| 16.39 | | | | | | | | 1.00 |

| RRT/AP | 6.71 | 7.65 | 8.18 | 8.98 | 9.49 | 11.3 | 11.52 | 16.38 |
|---|---|---|---|---|---|---|---|---|
| 6.71 | 1.00 | 1.14 | 1.22 | 1.34 | 1.41 | 1.68 | 1.72 | 2.44 |
| 7.65 | | 1.00 | 1.07 | 1.17 | 1.24 | 1.48 | 1.51 | 2.14 |
| 8.18 | | | 1.00 | 1.10 | 1.16 | 1.38 | 1.41 | 2.00 |
| 8.98 | | | | 1.00 | 1.06 | 1.26 | 1.28 | 1.82 |
| 9.49 | | | | | 1.00 | 1.19 | 1.21 | 1.73 |
| 11.3 | | | | | | 1.00 | 1.02 | 1.45 |
| 11.52 | | | | | | | 1.00 | 1.42 |
| 16.38 | | | | | | | | 1.00 |

A250 can also be characterized by its IC50 value determined in cancer cell line assays. An average IC50 value of the produced A250 in PA-1 human ovarian cancer cell line is 25 mcg/ml.

A250 shows significant anti-cancer efficacy against cancer cell lines (TABLE 3.). It is a surprising observation that the activities of A250 samples are very similar independently the place of origin of the corresponding FWGE.

TABLE 3
IC50 values (mcg/ml) of A250 produced from different industrial batches
of FWGE, manufactured in different countries, in different manufacturing
plats, at different time. Data for A2-NWS and A250-NSB are also shown.

| | A250- | A250- | A250- | A250- | A250- | A250- | A250- | A250- | A2- | A250- |
|---|---|---|---|---|---|---|---|---|---|---|
| | c73 | c74 | c73 | u1 | c74 | c73 | u2 | u3 | NWS | NSB |
| ¹PA-1 | 17.5 | 13.73 | 13.28 | 19.44 | 17.86 | 12.55 | 15.01 | 11.02 | 27.67 | ⁴NA |
| ²PC-3 | 59.93 | 65.81 | 68.07 | 75.73 | 72.35 | 65.50 | 90.53 | 69.45 | 69.78 | NA |
| ³B16- | 31.45 | 21.73 | 11.63 | 16.02 | 13.43 | 14.43 | 14.63 | 28.96 | 13.89 | NA |
| F10 | | | | | | | | | | |

¹Human ovarian cancer;
²Human prostate cancer;
³Murine melanoma;
⁴No activity.

A250 significantly reduced tumor progression and lengthen overall survival in experimental cancer studies. (See Example-7)

A250 has a favorable toxicity profile. (See Example-6)

A250 inhibits a great variety of kinases, which play important roles in the development and progression of human cancers and other diseases and physiological conditions. (See Example-8)

A250 can easily be produced in the laboratory and also on industrial scale. The A250 manufacturing process disclosed in the present invention use only such solvents, which are generally permitted in food technology. (See Example-2, 3, 4)

Uses of A250: A250 on its own, or in combination with other products, could be administered orally, and/or intraperitonally (ip), and/or intravenously (iv) to mammals suffering from neoplastic diseases, such as cancer, or having immune imbalances, such as autoimmune diseases, or having metabolic imbalances, such as metabolic syndrome. It may be used as a disease-preventative product, too. A250 could also be used for the standardization and/or quality control of wheat germ ferments/fermented wheat germ extracts and products containing said ferments/extracts.

Other active fractions could also been obtained from the fermented wheat germ extract's fractions. The mentioned A2KLID fraction is dissolved in 5-times quantity of methanol (w/v) and, water was given in 7-times quantity. The mixture is shaken, and the same quantity of chloroform, as the quantity of the methanol, is added. The sub-fraction (A2KLIDZW) remaining in the water phase is separated by centrifugation, the aqueous fraction is filtered through the Waters Oasis SPE column and, the adsorbed material is eluted by 100% methanol. The methanolic solution is dried. The resulting material has a honey-like state and a dark color (A2KLIDZWS). The chloroformic phase has a reddish color. The chloroform solution is evaporated, and the resulting material is dried with diisopropyl ether (“red material”, A2KLIDZKP or PA powder). A black, oily fraction, denoted as A2KLIDZF, is also isolated. This sub-fraction—also called as “black material” or FA—cannot not be dissolved into the water phase nor into the chloroformic phase but, could be dissolved in methanol.

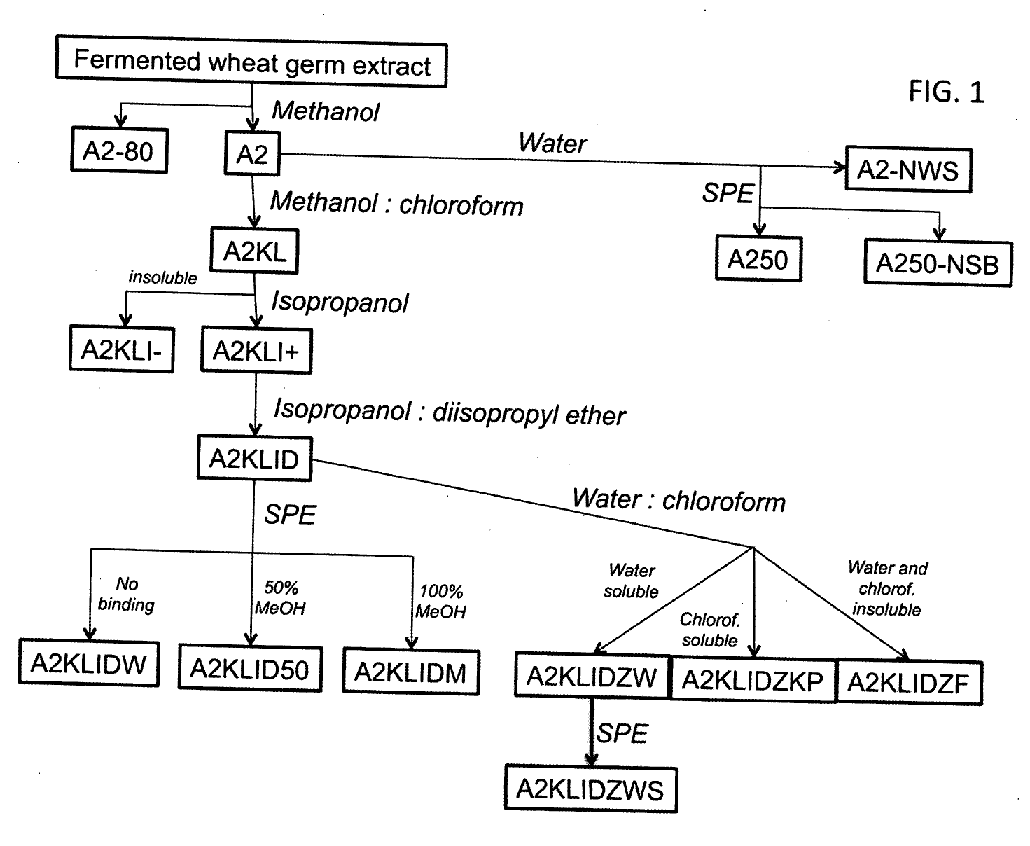

Isolation of A2KLIDZF: A2KLID is suspended in a relatively large amount of water. A part of the material could not be dissolved. The solution is filtered, and the filtered-out material, together with the water-insoluble material, are dissolved in methanol. The methanol solution is evaporated, and the resulting material is dried with diisopropyl ether (“black material”, A2KLIDZF, FA powder). The isolation chart of the fractions is shown in FIG. 1.

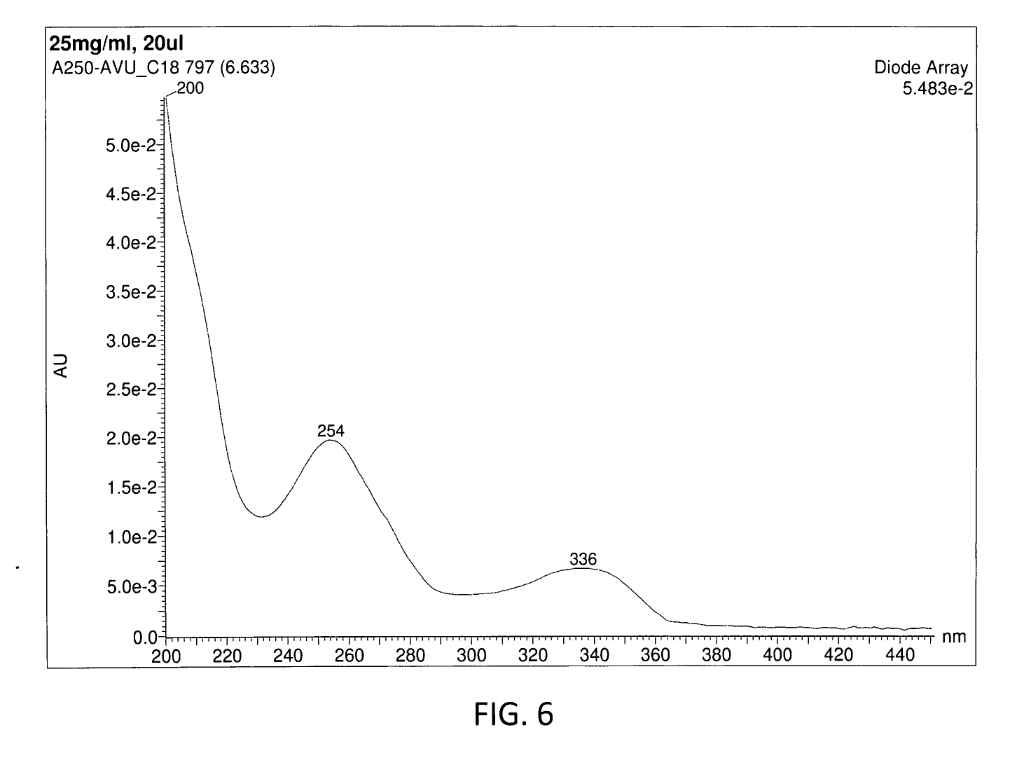

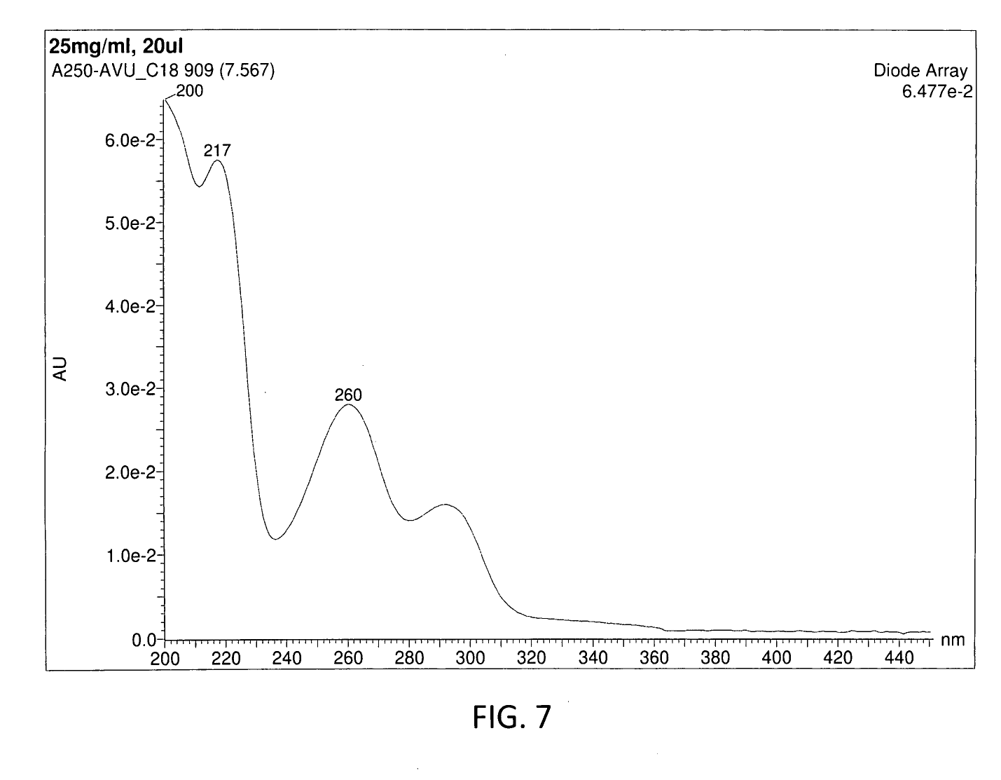

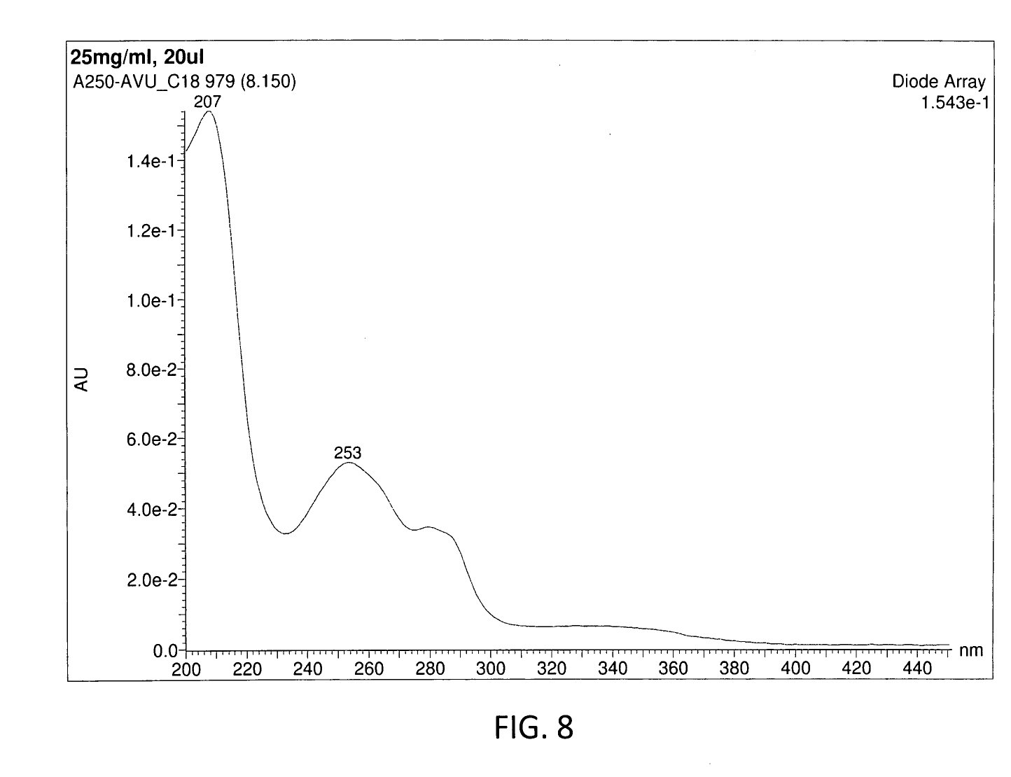

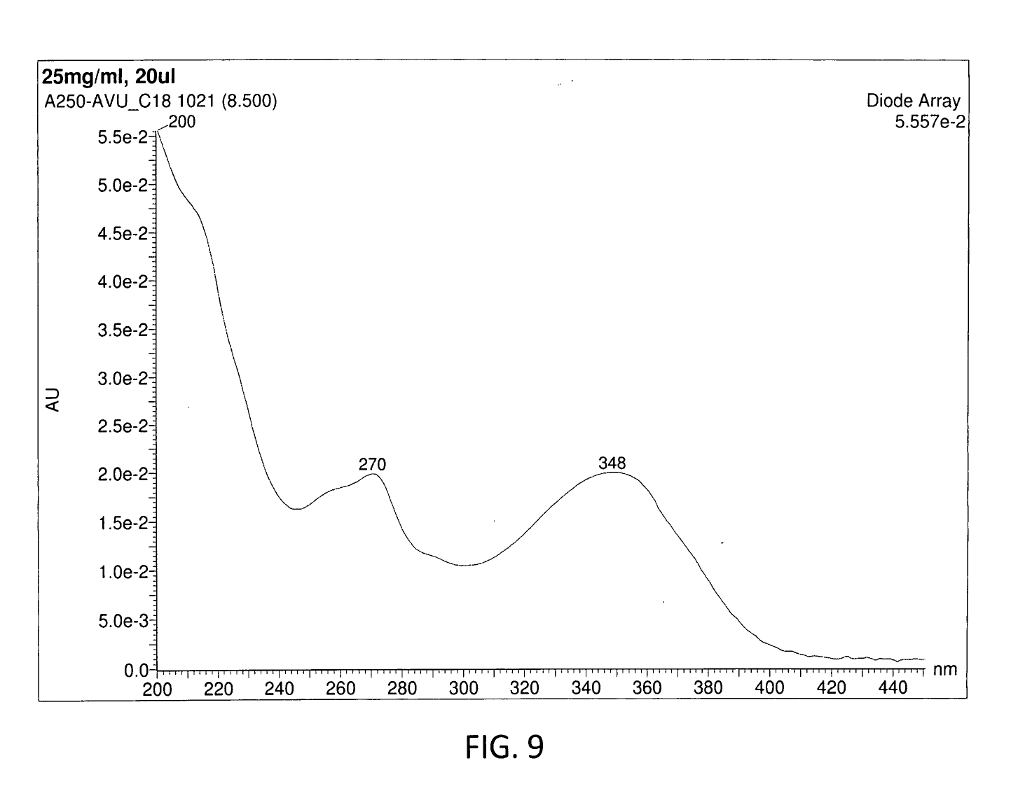

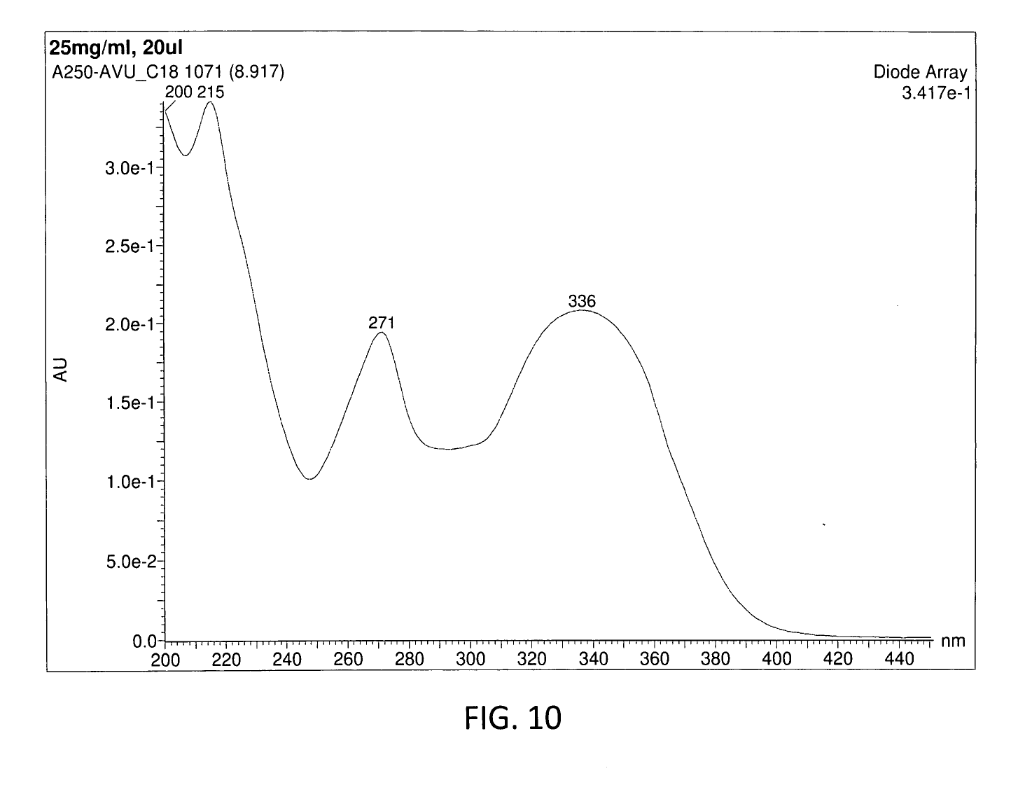

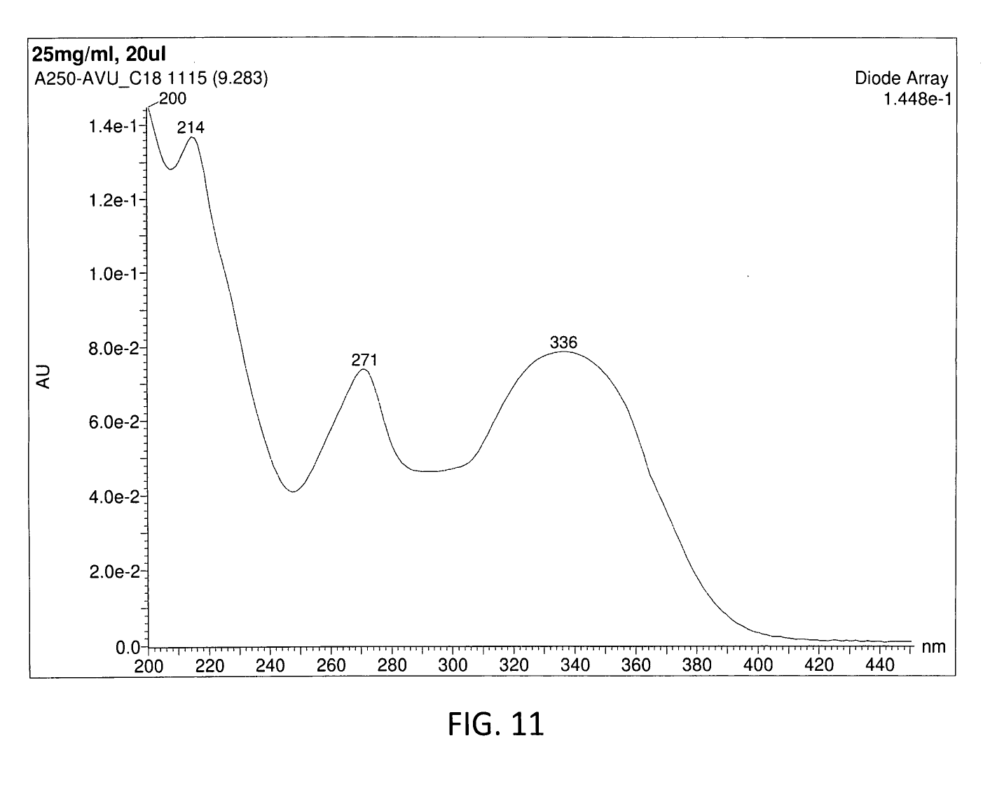

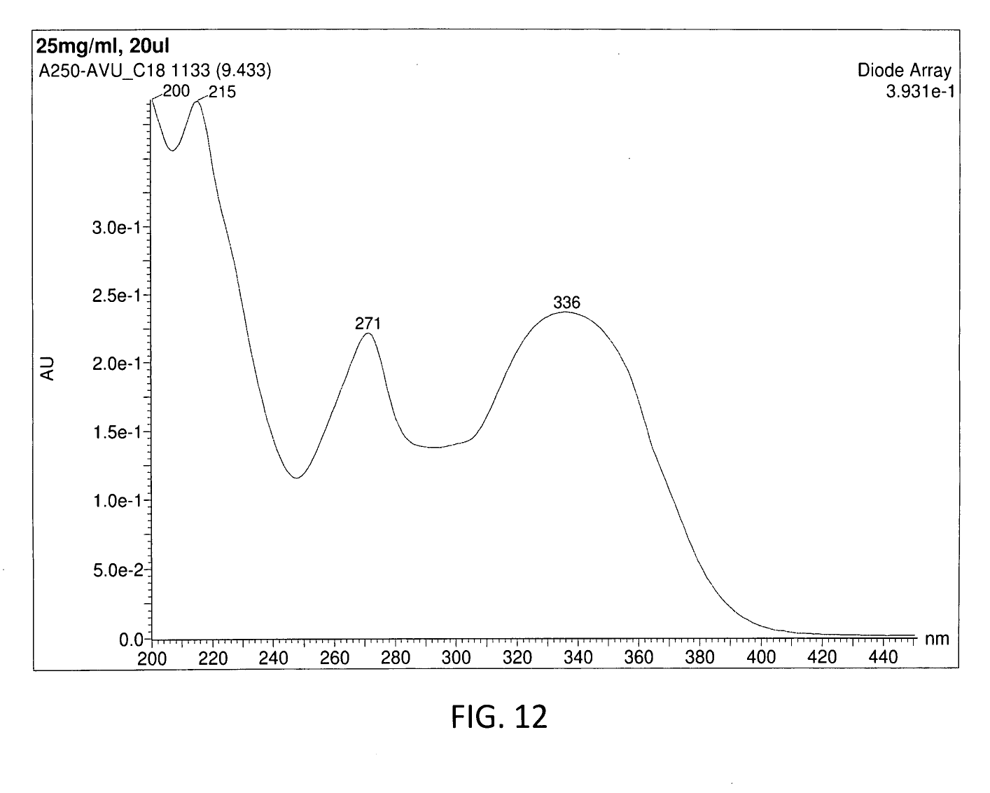

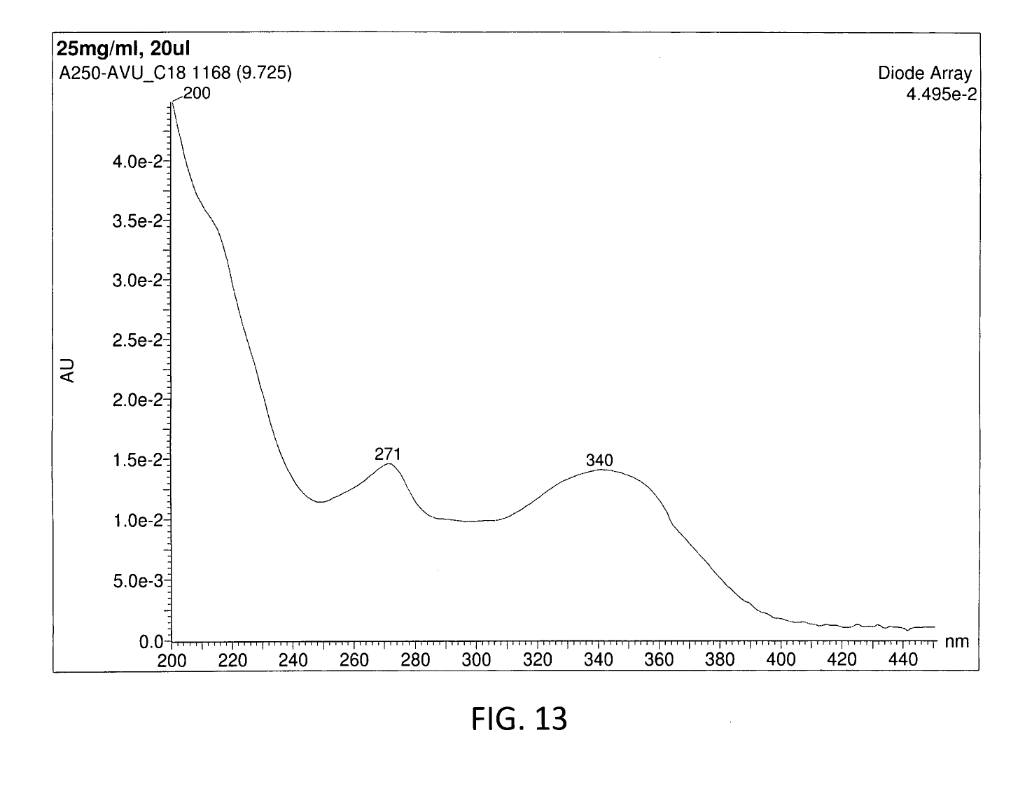

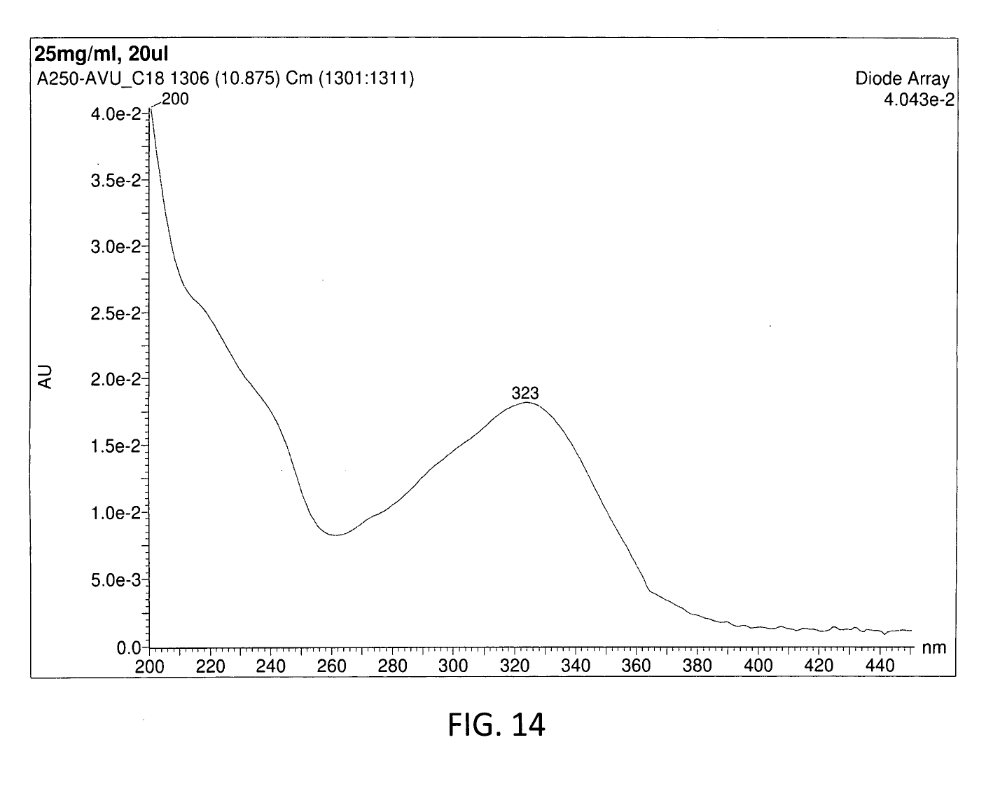

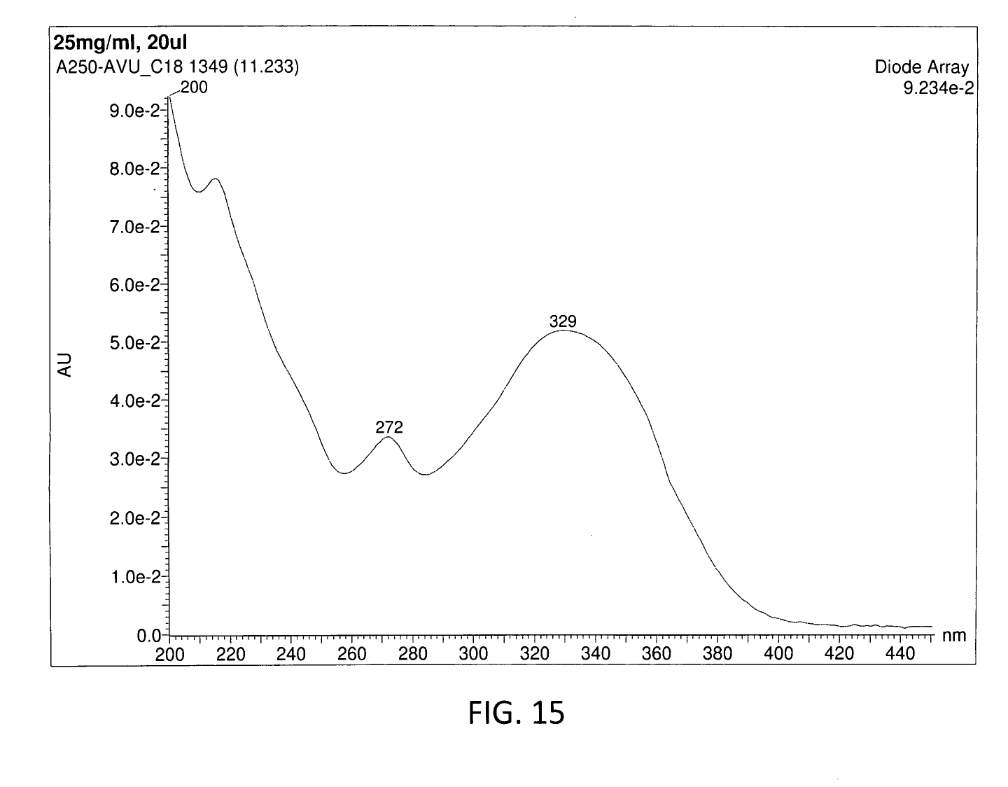

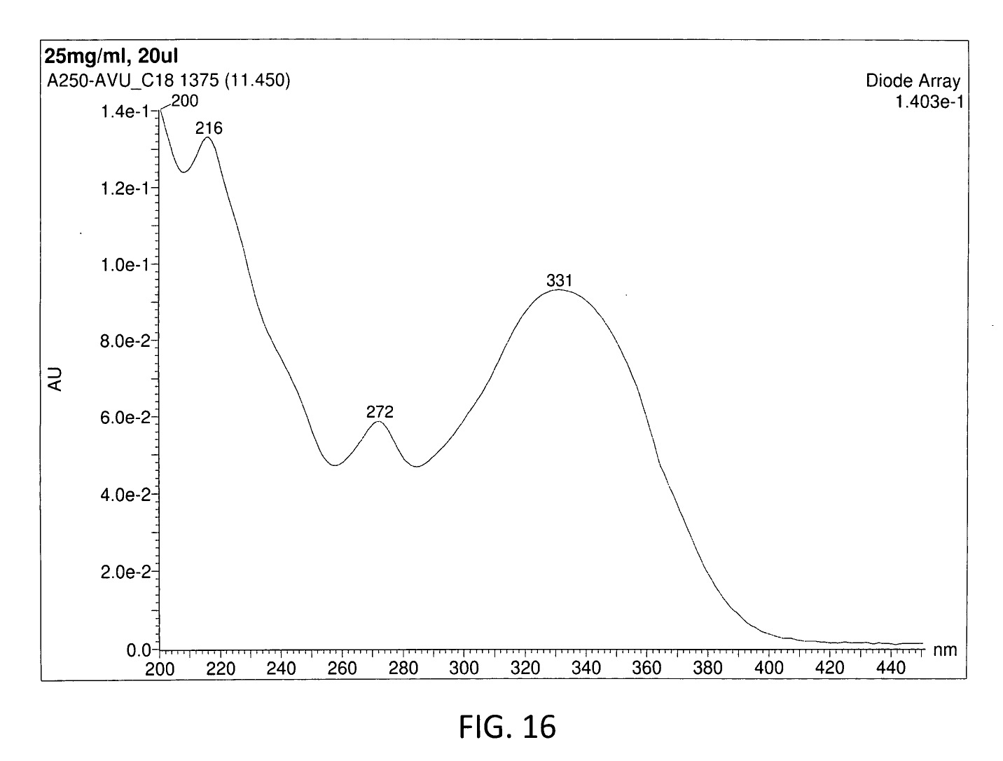

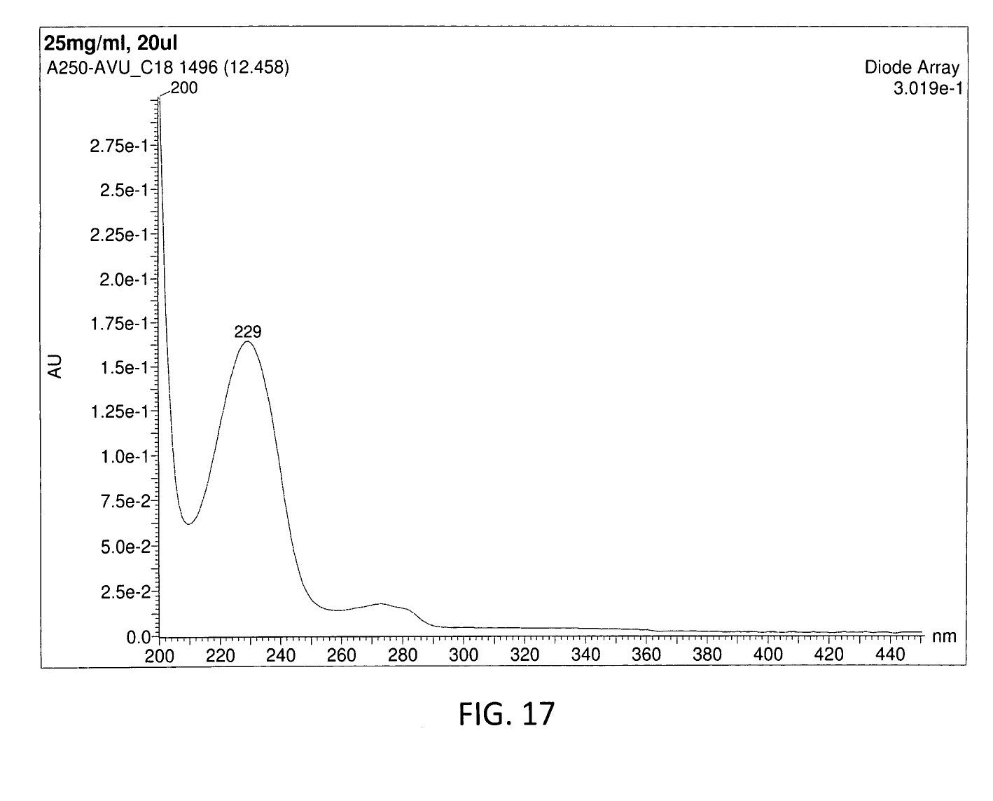

In particular, the disclosure teaches the following:
1. Biologically active fractions obtained from wheat germ ferment/fermented wheat germ extract.
2. Fractions, A250, A2-80, A2-NWS, A250-NSB, A2KL, A2KLI−, A2KLI+, A2KLID, A2KLIDW, A2KLID50, A2KLIDM, A2KLIDZW, A2KLIDZKP, A2KLIDZF, A2KLIDZWS, obtained by fractionation of wheat germ ferment/fermented wheat germ extract, obtained by fermenting wheat germ in aqueous medium in the presence of Saccharomyces cerevisiae, and by concentrating and/or dehydrating the fermentation liquid.
3. Fraction A250, obtained from wheat germ ferment/fermented wheat germ extract according to subparagraph 1.
4. Fraction A250, according to subparagraph 3, the HPLC fingerprint chromatogram of which basically corresponds to that in FIGS. 2, 3 and 4, and the UV-spectrums of which, detected at various times, basically correspond to those in FIGS. 6, 7, 8, 9, 10, 11, 12, 13, 14, 15, 16 and 17.
5. Fraction A250, obtained from wheat germ ferment/fermented wheat germ extract according to subparagraph 2.
6. Fraction A250, according to subparagraph 5, the HPLC fingerprint chromatogram of which basically corresponds to that in FIGS. 2, 3 and 4, and the UV-spectrums of which, detected at various times, basically correspond to those in FIGS. 6, 7, 8, 9, 10, 11, 12, 13, 14, 15, 16 and 17.
7. Process for the preparation of fraction A250 according to subparagraphs 3, 4, 5 and 6, from wheat germ ferment/fermented wheat germ extract, characterized by that the wheat germ ferment/fermented wheat germ extract is dissolved in alcohol, filtered, the filtrate is cooled down, filtered, the resulting alcohol solution is evaporated, the resulting dry material is dissolved in water, separated by solid phase extraction, the stationary phase is eluted by alcohol, and the alcohol solution is dried.
8. Process for the preparation of fraction A250 according to subparagraphs 3, 4, 5 and 6, from wheat germ ferment/fermented wheat germ extract, characterized by that the wheat germ ferment is dissolved in alcohol, filtered, the filtrate is evaporated, the resulting dry material is dissolved in water, separated by solid phase extraction, the stationary phase is eluted by alcohol, and the alcohol solution is dried.
9. Process for the preparation of fraction A250 according to subparagraphs 3, 4, 5 and 6, from wheat germ fermentation broth concentrate, characterized by that alcohol is mixed into the concentrate, the mixture is filtered, the alcohol is removed from the filtrate, the resulting aqueous phase is filtered and separated by solid phase extraction, the stationary phase is eluted by alcohol, and the alcohol solution is dried.
10. Process for the preparation of fraction A250 according to subparagraphs 3, 4, 5 and 6, characterized by that as solvent alcohol, preferably methanol or ethanol, more preferably, methanol is used.
11. Process for the preparation of fraction A250 according to subparagraphs 3, 4, 5 and 6, characterized by that as stationary phase for solid phase extraction a polymer, preferably silicon, more preferably, silicon with carbon chains is used.
12. Process for the preparation of fraction A250 according to subparagraphs 3, 4, 5 and 6, characterized by that as eluent for solid phase extraction alcohol, preferably methanol or ethanol, more preferably, methanol is used.
13. Process for the preparation of fraction A250 according to subparagraphs 3, 4, 5 and 6, characterized by that the drying is carried out by vacuum-evaporation, preferably by vacuum-drying, more preferably by vacuum-drying combined with lyophilization.
14. Previously undescribed biologically active molecules.
15. Previously undescribed biologically active molecules of natural and/or semi-synthetic origin.
16. Previously undescribed biologically active molecules, obtained from wheat germ ferment.
17. Previously undescribed biologically active molecules, obtained from wheat germ ferment, obtained by fermenting wheat germ in aqueous medium in the presence of Saccharomyces cerevisiae, and by concentrating and/or dehydrating the fermentation liquid.
18. Previously undescribed biologically active molecules, obtained from fraction A250.
19. Previously undescribed biologically active molecules, obtained from any of the fractions A2-80, A2-NWS, A250-NSB, A2KL, A2KLI−, A2KLI+, A2KLID, A2KLIDW, A2KLID50, A2KLIDM, A2KLIDZW, A2KLIDZKP, A2KLIDZF, A2KLIDZWS.
20. Molecules according to subparagraphs 14, 15, 16, 17, 18 and 19, characterized by that the biological activity is manifested in anti-cancer effect.
21. Molecules according to subparagraph 14, 15, 16, 17, 18 and 19, characterized by that the biological activity is manifested in immune-modulatory effect.
22. Molecules according to subparagraph 14, 15, 16, 17, 18 and 19, characterized by that the biological activity is manifested in metabolism modulatory effect.
23. Previously undescribed combinations of biologically active molecules.
24. Previously undescribed combinations of biologically active molecules of natural and/or semi-synthetic origin.
25. Previously undescribed combinations of biologically active molecules, obtained from wheat germ ferment/fermented wheat germ extract.
26. Previously undescribed combinations of biologically active molecules, obtained from wheat germ ferment/fermented wheat germ extract, obtained by fermenting wheat germ in aqueous medium in the presence of Saccharomyces cerevisiae, and by concentrating and/or dehydrating the fermentation liquid.
27. Previously undescribed combinations of biologically active molecules, obtained from fraction A250.
28. Previously undescribed combinations of biologically active molecules, obtained from any of the fractions A2-80, A2-NWS, A250-NSB, A2KL, A2KLI−, A2KLI+, A2KLID, A2KLIDW, A2KLID50, A2KLIDM, A2KLIDZW, A2KLIDZKP, A2KLIDZF, A2KLIDZWS.
29. Combination of molecules according to subparagraphs 23, 24, 25, 26, 27 and 28, characterized by that the biological activity is manifested in anti-cancer effect.
30. Combination of molecules according to subparagraphs 23, 24, 25, 26, 27 and 28, characterized by that the biological activity is manifested in immune-modulatory effect.
31. Combination of molecules according to subparagraphs 23, 24, 25, 26, 27 and 28, characterized by that the biological activity is manifested in metabolism modulatory effect.
32. Preparation containing as active ingredient one or more fractions according to subparagraphs 1, 2, 3, 4, 5 and 6.
33. Preparation according to subparagraphs 1, 2, 3, 4, 5 and 6, characterized by that the active ingredient is formulated in forms of tablets, coated tablets, dragées, granules, sachets, capsules, solution, suspension, emulsion, spray, suppository, ointment, patch, liposome.
34. Preparation according to subparagraphs 3, 4, 5 and 6, containing as active ingredient fraction A250.
35. Preparation according to subparagraphs 3, 4, 5 and 6, characterized by that the fraction A250 is formulated in forms of tablets, coated tablets, dragées, granules, sachets, capsules, solution, suspension, emulsion, spray, suppository, ointment, patch, liposome.
36. Preparation according to subparagraphs 14, 15, 16, 17, 18 and 19, containing as active ingredient one or more molecules.
37. Preparation according to subparagraphs 14, 15, 16, 17, 18 and 19, characterized by that the active ingredient is formulated in forms of tablets, coated tablets, dragées, granules, sachets, capsules, solution, suspension, emulsion, spray, suppository, ointment, patch, liposome.
38. Preparation according to subparagraphs 23, 24, 25, 26, 27 and 28, containing as active ingredient combination of molecules.
39. Preparation according to subparagraphs 23, 24, 25, 26, 27 and 28, characterized by that the active ingredient is formulated in forms of tablets, coated tablets, dragées, granules, sachets, capsules, solution, suspension, emulsion, spray, suppository, ointment, patch, liposome.
40. Use of fractions according to subparagraphs 1, 2, 3, 4, 5 and 6, for the production of preparations having anticancer and/or immune-modulatory and/or metabolism modulatory properties.
41. Use of fractions according to subparagraphs 1, 2, 3, 4, 5 and 6, for the production of dietary supplement, medical food or dietary food for special medical purpose, herbal medicine, drug for mammals, respectively.
42. Use of fraction A250 according to subparagraphs 3, 4, 5 and 6, for the production of preparations having anticancer and/or immune-modulatory and/or metabolism modulatory properties.
43. Use of fraction A250 according to subparagraphs 3, 4, 5 and 6, for the production of dietary supplement, medical food or dietary food for special medical purpose, herbal medicine, drug for mammals, respectively.
44. Use of molecules according to subparagraphs 14, 15, 16, 17, 18 and 19, for the production of preparations having anticancer and/or immune-modulatory and/or metabolism modulatory properties.
45. Use of molecules according to subparagraphs 14, 15, 16, 17, 18 and 19, for the production of dietary supplement, medical food or dietary food for special medical purpose, herbal medicine, drug for mammals, respectively.
46. Use of combination of molecules according to subparagraphs 23, 24, 25, 26, 27 and 28, for the production of preparations having anticancer and/or immune-modulatory and/or metabolism modulatory properties.
47. Use of combination of molecules according to subparagraphs 23, 24, 25, 26, 27 and 28, for the production of dietary supplement, medical food or dietary food for special medical purpose, herbal medicine, drug for mammals, respectively.
48. Method of treatment and/or prevention of cancer, and/or prevention of immunological diseases, and/or prevention of metabolic imbalances, characterized by administering to the patient an effective amount of the preparation or preparations containing one or more of the fractions according to subparagraphs 1, 2, 3, 4, 5 and 6.
49. Method of treatment and/or prevention of cancer, and/or prevention of immunological diseases, and/or prevention of metabolic imbalances, characterized by administering to the patient an effective amount of the preparation or preparations containing the fraction A250 according to subparagraphs 3, 4, 5 and 6.
50. Method of treatment and/or prevention of cancer, and/or prevention of immunological diseases, and/or prevention of metabolic imbalances, characterized by administering to the patient an effective amount of the preparation or preparations containing one or more of the molecules according to subparagraphs 14, 15, 16, 17, 18 and 19.
51. Method of treatment and/or prevention of cancer, and/or prevention of immunological diseases, and/or prevention of metabolic imbalances, characterized by administering to the patient an effective amount of the preparation or preparations containing one or more of the molecular combinations according to subparagraphs 23, 24, 25, 26, 27 and 28.
52. Treatment and/or prevention of cancer, and/or immunological diseases, and/or metabolic imbalances, characterized by administering to the patient an effective amount of the preparation or preparations containing one or more of the fractions according to subparagraphs 1, 2, 3, 4, 5 and 6.
53. Treatment and/or prevention of cancer, immunological diseases, metabolic imbalances, characterized by administering to the patient an effective amount of the preparation or preparations containing the fraction A250 according to subparagraphs 3, 4, 5 and 6.
54. Treatment and/or prevention of cancer, immunological diseases, metabolic imbalances, characterized by administering to the patient an effective amount of the preparation or preparations containing one or more of the molecules according to subparagraphs 14, 15, 16, 17, 18 and 19.
55. Treatment and/or prevention of cancer, immunological diseases, metabolic imbalances, characterized by administering to the patient an effective amount of the preparation or preparations containing one or more of the molecular combinations according to subparagraphs 23, 24, 25, 26, 27 and 28.
56. Process for the stimulation of immune functions or for the modulation of pathological immune functions, characterized by administering to the patient an effective amount of the preparation or preparations containing one or more of the fractions according to subparagraphs 1, 2, 3, 4, 5 and 6.
57. Process for the stimulation of immune functions or for the modulation of pathological immune functions, characterized by administering to the patient an effective amount of the preparation or preparations containing the fraction A250 according to subparagraphs 3, 4, 5 and 6.
58. Process for the stimulation of immune functions or for the modulation of pathological immune functions, characterized by administering to the patient an effective amount of the preparation or preparations containing one or more of the molecules according to subparagraphs 14, 15, 16, 17, 18 and 19.
59. Process for the stimulation of immune functions or for the modulation of pathological immune functions, characterized by administering to the patient an effective amount of the preparation or preparations containing one or more of the molecular combinations according to subparagraphs 23, 24, 25, 26, 27 and 28.
60. Use of one or more fractions according to subparagraphs 1, 2, 3, 4, 5 and 6, for the standardization of fermented wheat germ extracts.

Drawing in, the disclosure also teaches the following

### EXAMPLES

Further particulars of the invention are described in the examples, without limiting the invention to the examples.

### Example-1

### Determination of Enrichment of In Vitro Anti-Cancer Activity in Fermented Wheat Germ Extract Fractions

Activity enrichment in a certain FWGE fraction is defined as the quotient of the IC50 (mcg/ml) value of FWGE and that of its given fraction, respectively. The higher the value, the better the enrichment. (Note. Values can only be compared if come from the same IC50 measurement session.) IC50 values are obtained in PA-1 and/or OVCAR-5 human ovarian cancer cell lines by MTT Cell Proliferation Assay. The test material is prepared as follows: 3 mg of the fraction is dissolved in 100 microliter of ethanol, and is diluted with 900 microliter of medium (Dulbecco's Modified Eagle Medium, DMEM). The resulting solution is further diluted by a factor of 4× during the test, thus the originally added ethanol has no effect on the biological results.

Activity enrichment (Measurement Session 1 whereas A2: 2.8 means fraction A2 is 2.8 times more active than FWGE):

### A2: 2.8

### A2KLI−: 4.8

### A2KLIDM: 20

### A2KLID50: 27

### A2KLIDW: 1.5

### A250: 20

### Activity Enrichment (Session 2):

### A2: 3.4

### A2-80: 1.3

### Example-2

### Laboratory Production of Compound A250

30 g A2 is dissolved in 1600 ml of water. The solution is filtered through a 0.2 micron sterile filter (the undissolved material, denoted as A2-NWS, is collected) and, the filtered solution is flushed through the above mentioned Waters Oasis adsorbent. The column is washed with water and, a fraction denoted as A250-NSB is obtained. After this step, the column is washed with 100% methanol and, the methanolic solution is dried. The yield of the dark red powder (A250) is about 6-7% of the A2 fraction and, about 2.5-3.5% of the original fermented wheat germ extract (based on dry matter content).

### Example-3

### Industrial Production (1) of A250

In a mixed tank reactor, 150 kg of fermented wheat germ extract powder—manufactured according to the patent Hidvegi (PCT HU1000025)—and 350 liters of 100% methanol are mixed and the mixture is filtered. This step should be repeated twice with the filtered-out precipitate. The united methanolic filtrates are cooled down to −80 degrees Celsius and, pumped through a filter, preferably hollow fiber filter tubes, of 10 micron nominal pore size, to remove the cold precipitate from the mixture. The methanolic filtrate is then evaporated under vacuum to dryness. About 50 kg dry material (A2) is resulted. This material is dissolved in 500 liters of water and, separated by SPE: stationary phase is a polymer, preferably silicon, more preferably, silicon with carbon chains. The solid phase is washed with water and eluted by 3-times more methanol than the volume of the solid phase column. The resulted methanol solution is vacuum-evaporated to dryness. The quantity of the A250 fraction is about 4.5 kg.

### Example-4

### Industrial Production (2) of A250

In a more economical way, crude A250 could directly be produced from the cell-free filtered fermentation broth containing about 20% fermented wheat germ extract solids. Mix 850 liters of 100% methanol into a reactor containing about 680 liters of said fermented wheat germ extract concentrate and pump the mixture through a 0.2 micron filter. Remove methanol from the filtrate by vacuum-destillation, push the aqueous phase through a 10 micron filter and separate the expected 6 kg of crude A250 from the filtered solution by SPE similarly as described in Example-2.

### Example-5

### Analysis of A250 Contents in Products Containing Fermented Wheat Germ Extract

500 mg sample is suspended into 5 ml of 100% methanol. The mixture is sonicated for 5 minutes, then stirred/shaken. This dissolution process is repeated for 2 more times. The solution is centrifuged (10 min, 4000 rpm) and, the supernatant is transferred into a rotary vacuum evaporator and dried at a temperature not exceeding 30 degrees of Celsius. In the case of a poor vacuum supply at the evaporator, a small amount of water may remain in the concentrated solution, which may be dried in a freeze-drier. The yield of A2 is about 200-210 mg. A2 is dissolved in 10 ml of water, and the solution is filtered through a 0.2-0.45 micron syringe. The filtrate is injected onto an SPE cartridge (Waters Oasis HB). Prior to usage, the cartridge is first washed with 100% methanol, then 50% methanol and finally, is equilibrated with water. After SPE separation, the stationary phase is washed with water, the volume of which is 3-times of the volume of the cartridge, and eluted by 100% methanol, the volume of which is 3-6-times of the volume of the cartridge. The eluted methanolic solution is vacuum evaporated to dryness at a temperature not exceeding 30 degrees of Celsius. Yield of A250 is 13-15 mg. For HPLC fingerprint assay, A250 is dissolved in DMSO in a final concentration of 25 mg/ml. 20 microliter A250 solution is injected into the equipment with a flow rate of 2 ml/min. Detection is carried out at any or all of the following wavelengths: 254 nm, 269 nm, 280 nm, 291 nm.

### Example-6

### In Vivo Toxicological Study of A250

Safety study of the fraction A250, obtained as described in Example 2 of the present invention, was investigated in a 15 days long toxicology experiment with intravenous (iv) treatment. Prior to the experimental work, the research protocol has been approved by the institutional review board (IRB).

Experimental animals: BALB/c mice.
Number of animals: 10 (2 animal/group).
Number of groups: 4+1 (control group).
Test material: A250-c7.
Treatment: intravenous injection once a day.
Days of injection: 1, 2, 6, 7, 8, 9, 12, 13, 14, 15.
Solvent: DMSO (5% final concentration)+Saline

### Groups and Doses:

| | |
|---|---|
| Group 1 | 43 mg/kg/day |
| Group 2 | 86 mg/kg/day |
| Group 3 | 215 mg/kg/day |
| Group 4 | 430 mg/kg/day |
| Group 5 | Saline + 2% DMSO |

The A250-c7 sample was dissolved in DMSO and diluted to the final concentration with saline. Before injection, all samples were sterile filtered with 0.22 mcm PTFE syringe filter. 200 mcl of test solution was injected into the tail vein of the animals.

Results. On day 6, in Group 4 one mouse died just after the injection because the animal accidentally got a lethal volume of the solvent (DMSO): 300 mcl of sample instead of the regular 200 mcl. From day 6 of the experiment, in Groups 3 and 4, inflammation could be seen at the site of injection. After day 12, group 4 was not treated any more since the size of the wound at the inflammation became larger. All mice in Groups 1, 2 and 3, and the second mouse in Group 4 survived the treatment without any toxic sign, which could be associated with the administration of A250.

Conclusion. It could be concluded that the intravenous administration of A250 remain safe.

### Example-7

### Anti-Tumor Effects of A250 in Sarcoma Mice

The comparative anti-tumor effects of A250, obtained as described in Example 2 of the present invention, and the lyophilized fermented wheat germ extract (denoted as A14) were investigated in S-180 sarcoma mice. A250 samples (denoted as U2 and C73, respectively) were produced from two, industrially manufactured FWGE batches of different origin. (One of the batches is the same as A14.) Anti-tumor effects of the samples were measured by their effects on tumor growth and on overall survival. Prior the experimental work, the research protocol has been approved by the institutional review board (IRB). Accepted standards of care for laboratory animals were strictly observed.

Groups and Doses:

A14: 2.0 g/kg/day; dissolved in water.

U2-1×: 0.036 g/kg/day; dissolved in DMSO.

U2-5×: 0.18 g/kg/day; dissolved in DMSO.

C73-1×: 0.036 g/kg/day; dissolved in DMSO.

C73-5×: 0.18 g/kg/day; dissolved in DMSO.

The dose of A14 (2.0 g/kg/day) is equivalent with the dose levels of FWGE used in previous in vivo research works. For the A250 groups, two doses were administered: the smaller dose of A250 (0.036 g/kg/day) is equivalent with the 60% of the FWGE dose, provided, that 3% is taken as the A250 content of A14. The larger dose of A250 (0.18 g/kg/day) represents a 5-fold dose increase.

Inbred SPF (specific pathogen free) female BDF1 mice, body weight 22-24 g, were used. Animals were given Altromin feed and tap drinking water ad libitum. S-180 murine tumor was transplanted. (Type: sarcoma. Origin: Chester Beatty Cancer Res. Inst., London, UK. Inoculum: tissue. Mode of transplantation: subcutaneous (s.c.). Host animal: BDF1 (C57B1 female X DBA/2 male) inbred hybrid mouse from SPF hygienic quality certified breed.) The transplantation of the tumor was carried out by s.c. transplantation of optimal tumor pieces and/or fragments into the interscapular region by tweezers. Prior to surgery, animals were narcotized by Nembutal (50 mg/kg, i.p.). Animals were treated orally once daily for 14 days (14×qd). Treatments were started after the appearance of the measurable tumor (7 days after tumor transplantation). After randomization, groups of seven animals each were formed. Randomization was carried out by measuring each animal's tumor volume thus, getting a mean value for tumor size. Mice, having larger or smaller tumor than that of the mean value, were discarded. At baseline, the average tumor volumes in the groups were equal.

Evaluation of anti-tumor effect. The anti-tumor effects of the samples were determined by comparing changes of tumor volume and measuring overall survival in the treated and non-treated (control) groups. Digital callipers were used for the continuous measurement of tumor volumes. The determination of tumor volume was done by using the following formula, accepted and used in the literature (Tomayko M M, Reynolds C P (1989): Determination of subcutaneous tumor size in athymic (nude) mice. Cancer Chemother Pharmacol. 24: 148-54):

V=D²×L×π/6

where V=tumor volume, D=shorter diameter, L=longer diameter.

Animals were observed daily, and measurements of tumor volume was done in every second day.

Statistical methods of evaluation of the anti-tumor effects. Comparison of control group with treated ones was performed by analysis of variance (ANOVA). The multiple comparisons were done by the Tukey's method. Tumor growth inhibition was calculated with tumor volumes measured on day 21 after tumor inoculation. The (1−T/C) % values are shown, where (T) and (C) are the tumor volumes (cm³, mean±SD) in the treated and the control group, respectively. Survival analyses were carried out by the Kaplan-Meier method. Comparisons of the overall survivals were done by log rank probe.

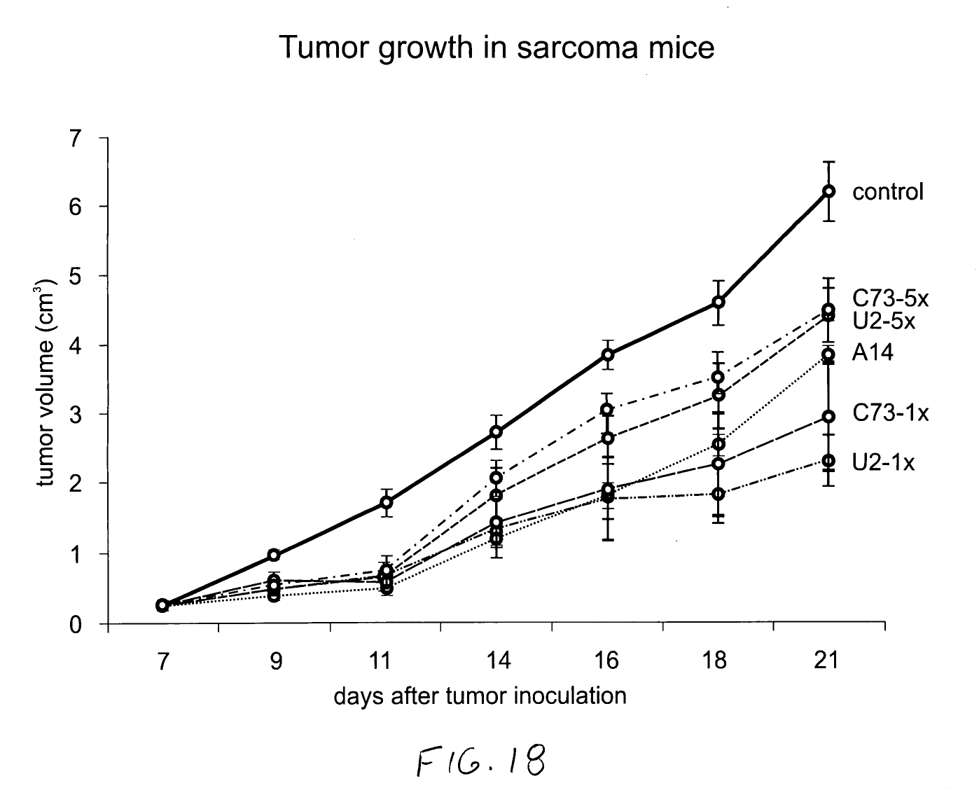

The results are shown in TABLES 4-9 and FIG. 18.

All of the test materials significantly inhibited sarcoma growth (TABLES 4 and 5; FIG. 18). The highest inhibition was achieved with the smaller doses of A250 (TABLE 6). The smaller doses of A250 were more efficient in terms of tumor growth inhibition (inhibition of disease progression) than both the larger doses of the corresponding compound and the whole FWGE.

TABLE 4
Tumor growth in sarcoma mice treated with FWGE and A250.

| | Days after tumor inoculation | | | | | | |
|---|---|---|---|---|---|---|---|
| | 7 | 9 | 11 | 14 | 16 | 18 | 21 |
| Control | 0.27 ± 0.02 | 0.95 ± 0.18 | 1.70 ± 0.53 | 2.73 ± 0.65 | 3.85 ± 0.55 | 4.60 ± 0.87 | 6.21 ± 1.16 |
| A-14 | 0.23 ± 0.03 | 0.38 ± 0.13 | 0.48 ± 0.22 | 1.20 ± 0.44 | 1.82 ± 0.49 | 2.56 ± 0.39 | 3.87 ± 0.28 |
| U2-1x | 0.22 ± 0.11 | 0.48 ± 0.20 | 0.68 ± 0.47 | 1.34 ± 1.14 | 1.78 ± 1.57 | 1.85 ± 1.15 | 2.33 ± 0.99 |
| U2-5x | 0.21 ± 0.03 | 0.47 ± 0.18 | 0.67 ± 0.20 | 1.82 ± 1.04 | 2.63 ± 0.92 | 3.24 ± 1.29 | 4.43 ± 1.02 |
| C73-1x | 0.24 ± 0.07 | 0.61 ± 0.26 | 0.55 ± 0.13 | 1.40 ± 0.93 | 1.92 ± 1.20 | 2.26 ± 1.97 | 2.97 ± 2.06 |
| C73-5x | 0.26 ± 0.11 | 0.52 ± 0.25 | 0.73 ± 0.50 | 2.07 ± 0.72 | 3.07 ± 0.90 | 3.55 ± 0.72 | 4.51 ± 0.50 |

TABLE 5
Comparison of tumor growth inhibition in treated groups with the
control on day 21 after tumor inoculation.

| | A-14 | U2-1x | U2-5x | C73-1x | C3-5x |
|---|---|---|---|---|---|
| Control | p < 0.001 | p < 0.001 | p < 0.01 | p < 0.01 | p < 0.01 |

TABLE 6
Tumor growth inhibition.

| | Tumor growth inhibition ratio (1-T/C) % | |
|---|---|---|
| Treatment | Mean | 95% CI |

| | | | |
|---|---|---|---|
| A-14 | 37.6 | 25.6 | 49.6 |
| U2-1x | 62.5 | 41.0 | 84.0 |
| U2-5x | 28.6 | 9.70 | 47.6 |
| C73-1x | 66.8 | 35.5 | 98.1 |
| C73-5x | 27.3 | 13.3 | 41.3 |

Similarly, all of the test materials significantly lengthened overall survival in sarcoma mice (TABLES 7 and 8). The highest physiological effect was achieved with the smaller doses of A250, i.e. the smaller doses of A250 were significantly more advantageous in terms of overall survival than the larger doses of the corresponding compound (TABLE 9). The smaller doses of A250 were also significantly more efficient in terms of overall survival than the whole FWGE (TABLE 9).

TABLE 7
Kaplan-Meier survival analysis.

Overall survival after tumor inoculation (days)

| Treatment | Mean | 95% CI | Median | 95% CI |
|---|---|---|---|---|
| Control | 19.6 | 18.1-21.0 | 18.0 | — |
| A14 | 26.9 | 23.7-30.0 | 28.0 | 17.7-38.3 |
| U2-1x | 32.3 | 30.8-33.8 | 33.0 | 30.7-35.6 |
| U2-5x | 25.7 | 24.1-27.4 | 26.0 | 23.7-28.3 |
| C73-1x | 33.0 | 31.7-34.3 | 33.0 | 30.4-35.6 |
| C73-5x | 28.7 | 27.2-30.2 | 29.0 | 23.9-34.1 |

Log Rank Statistics

TABLE 8
Comparison of treated groups with the control.

| | A-14 | U2-1x | U2-5x | C73-1x | C3-5x |
|---|---|---|---|---|---|
| Control | p < 0.01 | p < 0.001 | p < 0.001 | p < 0.001 | p < 0.001 |

TABLE 9
Pairwise comparison for each treated group.

| | A14 | U2-1x | U2-5x | C73-1x |
|---|---|---|---|---|
| U2-1x | p < 0.05 | | | |
| U2-5x | NS | p < 0.001 | | |
| C73-1x | p < 0.01 | NS | p < 0.001 | |
| C73-5x | NS | p < 0.01 | p < 0.05 | p < 0.05 |

Both tumor growth inhibition and overall survival results with A250 demonstrated the bell-shaped dose-response attribute as has been previously shown by the intact FWGE. Administration of FWGE and A250 remained safe. No toxic side-effects were seen.

### Example-8

### Kinase Panel and iTRAO Assay

A250, obtained as described in Example 2 of the present invention, shows significant effects on several important kinases. The results of a representative kinase panel assay with A250 is demonstrated in TABLE 10.

TABLE 10
Kinase activity changes upon A250 treatments.

| | A250 | | A250 | |
|---|---|---|---|---|
| | 0.04 mg/ml | | 0.004 mg/ml | |
| MKK1 | 53 | 10 | 34 | 18** |
| MKK2 | 54 | 6 | 41 | 9 |
| MKK6 | 83 | 4 | 96 | 7 |
| ERK1 | 77 | 7 | 126 | 14 |
| ERK2 | 71 | 17** | 95 | 8 |
| JNK1 | 51 | 6 | 81 | 3 |
| JNK2 | 76 | 5 | 94 | 6 |
| JNK3 | 64 | 9 | 91 | 5 |
| p38a MAPK | 137* | 3 | 99 | 22** |
| p38b MAPK | 71 | 10 | 100 | 1 |
| p38g MAPK | 79 | 1 | 111 | 7 |
| p38d MAPK | 82 | 3 | 96 | 2 |
| ERK8 | 15 | 8 | 57 | 1 |
| RSK1 | 17 | 2 | 67 | 10 |
| RSK2 | 4* | 1 | 26 | 4 |
| PDK1 | 90 | 5 | 117 | 5 |
| PKBa | 18 | 10 | 77 | 7 |
| PKBb | 1* | 1 | 4* | 1 |
| SGK1 | 81 | 14 | 82 | 1 |
| S6K1 | 29 | 1 | 84 | 2 |
| PKA | 82 | 5 | 103 | 1 |
| ROCK 2 | 60 | 2 | 98 | 11 |
| PRK2 | 85 | 7 | 91 | 3 |
| PKCa | 32 | 1 | 71 | 1 |
| PKCγ | 21 | 2 | 60 | 1 |
| PKCz | 78 | 4 | 107 | 7 |
| PKD1 | 3* | 0 | 5* | 0 |
| STK33 | 51 | 5 | 92 | 7 |
| MSK1 | 56 | 4 | 88 | 12 |
| MNK1 | 46 | 8 | 81 | 5 |
| MNK2 | 31 | 3 | 85 | 7 |
| MAPKAP-K2 | 10* | 1 | 81 | 1 |
| MAPKAP-K3 | 24 | 4 | 71 | 10 |
| PRAK | 10 | 1 | 88 | 2 |
| CAMKKb | 21 | 2 | 67 | 10 |
| CAMK1 | 8* | 3 | 5* | 1 |
| SmMLCK | 39 | 10 | 94 | 10 |
| PHK | 47 | 6 | 100 | 2 |
| DAPK1 | 46 | 1 | 96 | 13 |
| CHK1 | 121 | 6 | 95 | 5 |
| CHK2 | 26 | 1 | 71 | 2 |
| GSK3b | 9* | 1 | 63 | 3 |
| CDK2-Cyclin A | 37 | 6 | 72 | 14 |
| PLK1 | 78 | 3 | 90 | 1 |
| Aurora A | 25 | 5 | 77 | 1 |
| Aurora B | 52 | 16** | 107 | 2 |
| TLK1 | 94 | 7 | 114 | 1 |
| LKB1 | 58 | 2 | 79 | 12 |
| AMPK | 16 | 2 | 84 | 1 |
| MARK1 | 61 | 3 | 91 | 10 |
| MARK2 | 52 | 19** | 83 | 1 |
| MARK3 | 70 | 5 | 103 | 15 |
| MARK4 | 61 | 4 | 86 | 7 |
| BRSK1 | 26 | 4 | 70 | 4 |
| BRSK2 | 9* | 4 | 37 | 3 |
| MELK | 22 | 7 | 47 | 1 |
| NUAK1 | 11 | 1 | 81 | 3 |
| CK1 | 75 | 3 | 108 | 1 |
| CK2 | 3* | 1 | 38 | 8 |
| DYRK1A | 3* | 0 | 42 | 3 |
| DYRK2 | 3* | 0 | 20 | 2 |
| DYRK3 | 5* | 0 | 29 | 1 |
| NEK2a | 6* | 3 | 26 | 2 |
| NEK6 | 32 | 1 | 50 | 4 |
| IKKb | 42 | 1 | 93 | 3 |
| IKKe | 22 | 2 | 69 | 9 |
| TBK1 | 34 | 10 | 86 | 10 |
| PIM1 | 8* | 1 | 44 | 0 |
| PIM2 | 17 | 3 | 54 | 4 |
| PIM3 | 4* | 1 | 9* | 1 |
| SRPK1 | 12 | 4 | 54 | 2 |
| EF2K | 40 | 0 | 88 | 3 |
| EIF2AK3 | 58 | 4 | 83 | 13 |
| HIPK1 | 51 | 7 | 84 | 6 |
| HIPK2 | 35 | 1 | 35 | 0 |
| HIPK3 | 49 | 4 | 93 | 5 |
| CLK2 | 12 | 2 | 51 | 1 |
| PAK2 | 34 | 1 | 72 | 2 |
| PAK4 | 42 | 10 | 94 | 0 |
| PAK5 | 66 | 16** | 84 | 14 |
| PAK6 | 67 | 13 | 93 | 1 |
| MST2 | 34 | 25** | 78 | 12 |
| MST4 | 59 | 2 | 86 | 13 |
| GCK | 27 | 1 | 98 | 1 |
| MINK1 | 4* | 2 | 43 | 2 |
| MEKK1 | 39 | 5 | 97 | 17** |
| MLK1 | 24 | 0 | 92 | 7 |
| MLK3 | 11 | 0 | 33 | 7 |
| TAO1 | 66 | 15 | 60 | 1 |
| ASK1 | 107 | 5 | 109 | 2 |
| TAK1 | 149* | 2 | 106 | 28** |
| IRAK1 | 31 | 5 | 86 | 11 |
| IRAK4 | 43 | 5 | 105 | 16** |
| RIPK2 | 15 | 3 | 67 | 8 |
| OSR1 | 68 | 7 | 87 | 1 |
| TTK | 20 | 3 | 62 | 1 |
| MPSK1 | 75 | 0 | 111 | 8 |
| Src | 50 | 10 | 106 | 5 |
| Lck | 54 | 4 | 91 | 7 |
| CSK | 85 | 16** | 88 | 5 |
| YES1 | 64 | 9 | 111 | 1 |
| ABL | 64 | 6 | 90 | 4 |
| BTK | 10* | 0 | 32 | 2 |
| JAK2 | 55 | 25** | 98 | 3 |
| SYK | 72 | 6 | 89 | 7 |
| ZAP70 | 153* | 9 | 112 | 16** |
| TIE2 | 47 | 15 | 72 | 15 |
| BRK | 65 | 5 | 94 | 1 |
| EPH-A2 | 91 | 5 | 89 | 23** |
| EPH-A4 | 70 | 5 | 81 | 6 |
| EPH-B1 | 100 | 5 | 85 | 9 |
| EPH-B2 | 53 | 6 | 69 | 16** |
| EPH-B3 | 19 | 1 | 30 | 3 |
| EPH-B4 | 61 | 7 | 111 | 10 |
| FGF-R1 | 30 | 18** | 72 | 24** |
| HER4 | 46 | 7 | 58 | 8 |
| IGF-1R | 54 | 2 | 90 | 7 |
| IR | 36 | 1 | 51 | 7 |
| IRR | 56 | 2 | 106 | 7 |
| TrkA | 11 | 1 | 77 | 7 |
| VEG-FR | 12 | 6 | 74 | 4 |

(Bold numbers represent enzyme activity, regular numbers represent SD. A single asterisk (*) denotes activity value that represents significant change. A double asterisk (**) denotes an SD value that represents a vague result. Theoretically, an activity value of 100 represents no change. Activity values, which are smaller than 100, represent inhibition, while those which are over 100, represent activation of the corresponding kinases. Activation can simply be calculated by subtracting the value from 100, i.e. a value of 1 represents 99% inhibition. Similarly, a value of 137 represents 37% activation.)

Sixteen of 121 kinases were found to be significantly inhibited by A250. Inhibited kinases predominantly fall into 4 kinase family subclasses: CAMK (Calcium dependent kinases), CMGC (cyclin dependent kinases), TK (Tyrosine Kinases) and AGC (cAMP kinases), which all play important roles in human physiology, including the development and progression of neoplastic and other diseases.

For the quantitative proteomic analysis, 24 hours after plating, B16F10 cells were dosed with A250 in 80 ug/ml concentration. A250 was dissolved in culture medium (DMEM, Dulbecco's Modified Eagle Medium). One dish of cells were pelleted and washed with phosphate buffer after 4 h, 12 h and 24 h of incubation. iTRAQ (isobaric tag for relative and absolute quantitation) labeling was executed in accordance with the method described in Ross PL, et al. (2004): Multiplexed protein quantitation in Saccharomyces cerevisiae using amine-reactive isobaric tagging reagents. Mol Cell Proteomics 3:1154-69. After labeling, each solution was acidified by the addition of 3 ul trifluoroacetic acid (TFA), the separate fractions combined, and the total mixture dried in vacuo. The peptides were further analyzed using electrospray mass spectrometry per Bantscheff M, et al. (2008): Robust and sensitive iTRAQ quantification on an LTQ Orbitrap mass spectrometer. Mol Cell Proteomics. 7:1702-13.

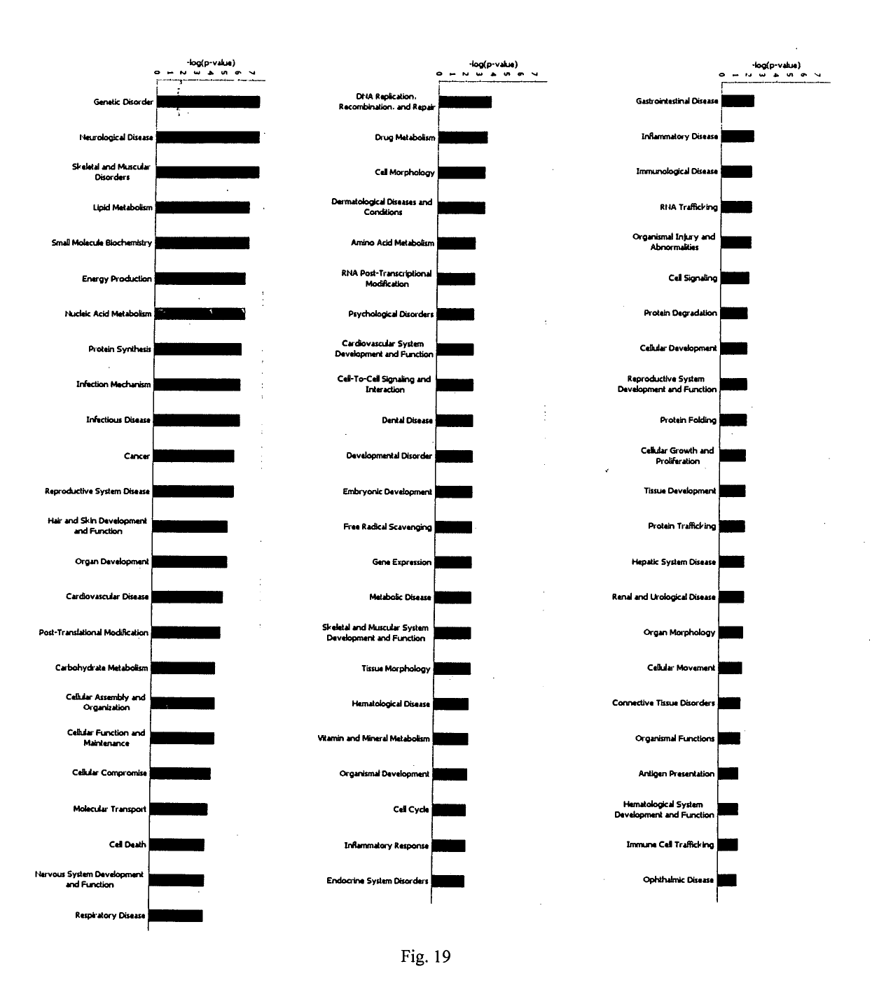

Significant results are shown in FIG. 19. FIG. 19 reflects the diseases and physiological functions influenced by the proteins which were significantly upregulated or downregulated by A250. It should be noted in particular that circadian clock functions were upregulated, although not shown in FIG. 19.

We claim:

1. A pharmacological formulation A250 having anti-cancer, immuno-modulatory, metabolic-regulatory, dietary supplement, and medical or dietary-food properties, wherein said formulation is obtained from fraction A2 of fermented wheat germ extract (FWGE), said fraction A2 being obtained by alcoholic extraction of said FWGE.

2. The formulation A250 of claim 1, wherein said formulation is characterized by its high-performance liquid chromatography (HPLC) fingerprint UV chromatogram.

3. The formulation A250 of claim 1, wherein said formulation is fabricated into a delivery modality.

4. A method of synthesizing a formulation A250 having anti-cancer, immuno-modulatory, metabolic-regulatory, dietary supplement, and medical or dietary food properties, wherein said formulation is obtained from fraction A2 of fermented wheat germ extract (FWGE), said fraction A2 being obtained by alcoholic extraction of said FWGE.

5. The formulation A250 of claim 4, wherein said formulation is obtained by the steps of
a. dissolving said fraction A2 in water,
b. separating by solid phase extraction (SPE).

6. The formulation A250 of claim 4, wherein said formulation is obtained by the steps comprising:
a. dissolving said FWGE in alcohol,
b. evaporating a resulting alcoholic solution,
c. dissolving the resulting dry material in water,
d. separating by solid phase extraction (SPE),
e. eluting the stationary phase and drying said resulting alcoholic solution.

7. The formulation A250 of claim 4, wherein said formulation A250 is obtained by mixing alcohol into wheat germ fermentation broth concentrate, filtering the mixture, removing the alcohol from the filtrate, filtering the resulting aqueous phase, separating by solid phase extraction (SPE), eluting the stationary phase by alcohol and drying a resulting alcoholic solution.

8. A method of synthesizing biologically active formulation A250 and fractions A2-80, A2-NWS, A250-NSB, A2KL, A2KLI−, A2KLI+, A2KLID, A2KLIDW, A2KLID50, A2KLIDM, A2KLIDZW, A2KLIDZWS, A2KLIDZKP and A2KLIDZP obtained from fraction A2 of fermented wheat germ extract (FWGE), including the steps of
a. Separating said formulation A250 or said fraction A2KL from said fraction A2, and
b. Fractionating said fraction A2KL to obtain fractions A2KLI−, A2KLI+, A2KLID, A2KLIDW, A2KLID50, A2KLIDM, A2KLIDZW, A2KLIDZWS, A2KLIDZKP and A2KLIDZP.

9. The formulation A250 of claim 6, wherein the eluent is alcohol.

10. The formulation A250 of claim 6, wherein said alcohol is methanol.

11. The formulation A250 of claim 6, wherein said method comprises the further steps of:
a. a first filtering,
b. cooling down the resulting filtrate, and
d. a second filtering.

12. The method of claim 4, wherein said formulation is used for standardizing manufacturing of said FWGE and products containing said FWGE.

13. The method of claim 5, further comprising the steps of
(c) eluting the stationary phase by alcohol and
(d) drying a resulting alcoholic extraction.
